(19) United States
(12) Patent Application Publication
(10) Pub. No.: US 2026/0108366 A1
(43) Pub. Date: Apr. 23, 2026

(54) Alignment System for Medical Devices

(71) Applicant: Bolt XT, Inc., Concord, MA (US)

(72) Inventors: Robert S. Brown, Lexington, MA (US); Patrick West, Concord, MA (US); Xiaowei Chen, Lexington, MA (US); Adam C. Jacobs, Hollis, NH (US); David James Hibbard, Bedford, NH (US)

(21) Appl. No.: 19/360,045

(22) Filed: Oct. 16, 2025

Related U.S. Application Data
(60) Provisional application No. 63/708,918, filed on Oct. 18, 2024.

(51) Int. Cl. A61F 2/46; A61B 34/10

(52) U.S. Cl. CPC A61F 2/4612; A61B 34/10; A61F 2/4657; A61B 2034/102; A61B 2034/105; A61B 2034/107; A61F 2/4611; A61F 2002/4632; A61F 2002/4663; A61F 2002/4687; A61F 2002/469; A61F 2002/4696

(57) ABSTRACT

An alignment system includes an electronic alignment device and an attachment apparatus. The attachment apparatus includes a cavity for receiving the electronic device, and a coupling apparatus for coupling the attachment apparatus to a medical device. The electronic alignment device is used to provide guidance in aligning the medical device. The electronic alignment device may send images to a secondary screen during a surgical procedure.

### CROSS-REFERENCE TO RELATED APPLICATIONS

This application claims the benefit of Provisional Patent Application Number 63/708,918 filed Oct. 18, 2014, and titled “Alignment System for Medical Devices,” which is incorporated by reference herein in its entirety.

### BACKGROUND

Precisely placing and orienting medical devices during surgical procedures is a critical challenge in modern medicine. Surgeons often need to insert instruments, implants, or other devices into specific anatomical locations with a high degree of accuracy. This precision is essential for achieving optimal surgical outcomes, minimizing complications, and ensuring patient safety.

In many surgical procedures, even small deviations from the intended placement or orientation of a medical device can have significant consequences. For example, in orthopedic surgeries such as joint replacements or spinal procedures, the exact positioning of implants may affect the patient's range of motion, stability, and long-term outcomes.

Several factors may contribute to the difficulty of achieving precise placement and orientation. These includes anatomical variations, limited visibility and soft tissue interference. While surgeons may utilize computer assisted planning and intra-operative imaging to facilitate precisely placing a medical device, this may be made more difficult in surgeries where the patient's anatomy may change positions during the procedure. For example, in shoulder surgery procedures the anatomical structures of the shoulder, such as the glenoid, may change their positions and throughout the procedure, making it more difficult to match planned entry points and trajectories for medical devices with real-time configurations of the anatomy.

There is a need in the art for a system and method that addresses the shortcomings discussed above.

### SUMMARY

In some aspects, the techniques described herein relate to an attachment apparatus for use with an electronic device for alignment of a medical device, the attachment apparatus including: a first wall and a second wall opposite the first wall; a cavity between the first wall and the second wall for receiving the electronic device; a coupling apparatus for coupling the attachment apparatus to the medical device; an opening on the second wall; and a reflector assembly for redirecting light entering through the opening.

In some aspects, the techniques described herein relate to an alignment system for aligning a medical device, including: an electronic device, the electronic device further including: a first side and a second side; a sensor on the first side of the electronic device; and a display on the first side of the electronic device; an attachment apparatus, the attachment apparatus further including: a first wall and a second wall; a cavity between the first wall and the second wall for receiving the electronic device; a coupling apparatus for coupling the attachment apparatus to the medical device; a first opening on the first wall for viewing the display of the electronic device; a second opening on the second wall; and a reflector assembly for redirecting light entering through the second opening to the sensor on the first side of the electronic device.

In some aspects, the techniques described herein relate to an electronic device, including: a processor; memory; a display disposed on a first side of the electronic device; a sensor disposed on the first side of the electronic device; the processor configured to: receive information from the sensor and use the information from the sensor to register the electronic device to an anatomical structure in a patient during a surgical procedure.

In some aspects, the techniques described herein relate to a method of using an alignment system, including: providing, at an electronic device, information about a designated trajectory for inserting a medical device into an anatomical structure of a patient; inserting the electronic device within an attachment apparatus and coupling the medical device to the attachment apparatus so that the electronic device and the medical device have a fixed relative orientation; registering the electronic device to the anatomical structure; adjusting an orientation of the electronic device until the electronic device indicates that the medical device has the designated trajectory relative to the anatomical structure.

In some aspects, the techniques described herein relate to a method, including: receiving, at an electronic device, a designated trajectory for a medical device relative to an anatomical structure; receiving, at the electronic device, 3D depth data; registering, using the 3D depth data, the electronic device to the anatomical structure; receiving, at the electronic device, gyroscopic information; determining, using the gyroscopic information, an orientation of the electronic device relative to the anatomical structure; and displaying, using the electronic device, information to guide a surgeon in aligning the medical device with the designated trajectory.

In some aspects, the techniques described herein relate to an attachment apparatus for use with an electronic device for alignment of a medical device, the attachment apparatus including: a first side portion and a second side portion; a cavity between the first side portion and the second side portion for receiving the electronic device; a coupling apparatus for coupling the attachment apparatus to the medical device; a first opening on the first side portion, wherein the first opening is configured so that a display of the electronic device is visible through the first opening and so that a first sensor of the electronic device can capture light through the first opening; and a second opening on the second side portion, wherein the second opening is configured so that a second sensor of the electronic device can capture light through the second opening.

In some aspects, the techniques described herein relate to an electronic device, including: a processor; memory; a first sensor disposed on a first side of the electronic device and a second sensor disposed on a second side of the electronic device; the processor configured to: receive information from the first sensor and use the information from the first sensor to perform an initial registration between the electronic device and an anatomical structure in a patient during a surgical procedure; and receive information from the second sensor and use the information from the second sensor to perform another registration between the electronic device and the anatomical structure in the patient during the surgical procedure.

In some aspects, the techniques described herein relate to a method of using a system including an attachment apparatus, an electronic device disposed within the attachment apparatus, and a medical device coupled to the attachment apparatus, including: positioning the system so that a first sensor on a first side of the electronic device is oriented towards an anatomical structure in a patient until the system indicates that the anatomical structure has been registered; rotating the system so that a second sensor on a second side of the electronic device is oriented towards the anatomical structure; and positioning the system to align the medical device with the anatomical structure while the second sensor is oriented towards the anatomical structure.

In some aspects, the techniques described herein relate to an alignment system for aligning a medical device with an anatomical structure in a patient, the alignment system including: an electronic device; an auxiliary sensor system configured to detect 3D depth data, wherein the auxiliary sensor system can communicate with the electronic device; and an attachment apparatus for receiving the electronic device and the auxiliary sensor system, wherein the attachment apparatus further includes a coupling apparatus for coupling the medical device to the attachment apparatus.

In some aspects, the techniques described herein relate to a method, including: receiving, at an electronic device, a designated trajectory for a medical device relative to an anatomical structure; receiving, at the electronic device, 3D depth data from one or more sensors of the electronic device; registering, using the 3D depth data, the electronic device to the anatomical structure; receiving, at the electronic device, sensor information from a sensor that is different than the one or more sensors; determining, using the sensor information, an orientation of the electronic device relative to the anatomical structure; establishing communication between the electronic device and a display device that is separate from the electronic device; sending, by the electronic device, image information to the display device, wherein the image information includes information to guide a surgeon in aligning the medical device with the designated trajectory.

Other systems, methods, features, and advantages of the disclosure will be, or will become, apparent to one of ordinary skill in the art upon examination of the following figures and detailed description. It is intended that all such additional systems, methods, features, and advantages be included within this description and this summary, be within the scope of the disclosure, and be protected by the following claims.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like reference numerals designate corresponding parts throughout the different views.

FIG. 1 is a schematic view of a process for using a medical alignment system to guide the placement (entry point) and trajectory (orientation) of a medical device inserted into a bone or other tissue at a surgical site.

FIG. 2 is a schematic view of a patient undergoing a shoulder replacement procedure.

FIGS. 3 and 4 are schematic views showing components of an alignment system, according to an embodiment.

FIG. 5 is a schematic view of some of the components of electronic device, according to an embodiment.

FIGS. 6-7 are schematic views showing the placement of external sensors, according to an embodiment.

FIG. 8 is a schematic perspective view of a first side of an apparatus with an electronic device, according to an embodiment.

FIG. 9 is a schematic view of a second side of an apparatus with an electronic device, according to an embodiment.

FIG. 10 is a schematic cross-sectional view of an alignment system, according to an embodiment.

FIG. 11 is a schematic view of a set of software modules that may be stored in memory and used by an electronic device, according to an embodiment.

FIG. 12 is a schematic view of a process for using a medical alignment system to guide the placement (entry point) and trajectory (orientation) of a guide pin that is inserted into the glenoid during a shoulder replacement surgery, according to an embodiment.

FIG. 13 is a schematic view showing a surgeon capturing a diagnostic image with an electronic device, according to an embodiment.

FIG. 14 is a schematic view showing a surgeon selecting a desired entry point and trajectory for inserting a guide pin into a glenoid, according to an embodiment.

FIG. 15 is a schematic view showing an augmented reality presentation of a surgical scene with a virtual guide pin, according to an embodiment.

FIGS. 16 and 17 are schematic views of another embodiment of an alignment system.

FIGS. 18 and 19 show steps of registering a surgical scene, according to an embodiment.

FIG. 20 is a schematic views of inputs to various software modules, according to an embodiment.

FIG. 21 is a schematic view of a process for using the exemplary alignment system to provide guidance for achieving the desired entry point and orientation of a medical device during a procedure, according to an embodiment.

FIG. 22 is a schematic view of some components of an alignment system including an auxiliary sensor system, according to an embodiment.

FIG. 23 shows another view of an alignment system with an auxiliary sensor system disposed within an attachment apparatus, according to an embodiment.

FIG. 24 shows a schematic view of the flow of data from different components of tan alignment system to various software modules running on the electronic device, according to an embodiment.

FIG. 25 is a schematic view of a scenario in which a surgeon uses a secondary display device with an alignment system, according to an embodiment.

FIG. 26 is a schematic view of an architecture for an electronic alignment device in communication with a secondary display screen.

FIG. 27 is a schematic view of a process for using an alignment system, according to an embodiment.

### DETAILED DESCRIPTION

The embodiments provide a medical alignment system (“alignment system”) that may be used to guide surgeons in finding the desired entry point and trajectory for a variety of different medical devices that may be inserted into an anatomical structure (such as bone or other tissue) during a surgical procedure. The alignment system includes an electronic alignment device (“electronic device”) and an attachment apparatus that facilitates coupling the electronic device to a medical device in a fixed relative orientation. The electronic device may then be used to measure information such as its location and/or orientation, and infer the associated location and orientation of the medical device based on knowing the fixed relative orientation (and location) of the medical device.

The electronic device includes sensors for mapping anatomical structures using 3D depth data. Using the 3D depth data, the electronic device may be registered to the anatomical structure so that the relative positions and orientations in space of the anatomical structure and of the electronic device may be compared in a common reference frame. Moreover, this registration between the electronic device and the anatomical structure allows designated or target entry point and trajectory information to be mapped to this same common reference frame. The electronic device further includes sensors, such as a gyroscope, for determining its own orientation as that orientation is changed relative to the anatomical structure during a surgical procedure. The electronic device further includes a display that may be used to provide a visual comparison between the current entry point/orientation of the medical device and the designated or target entry point/orientation determined in a pre-operative step. Furthermore, the process of registering the anatomical structure to the electronic device may be performed continuously (or at regular intervals), so that the relative entry point/orientation of the medical device compared to the designated entry point/orientation remains accurate at all times.

Electronic devices often incorporate optical sensors on both sides of the electronic device. In some cases, the sensors capable of gathering the highest resolution 3D depth data are located on the same side as the device's display. These ‘forward facing’ sensors are often used for applications such as facial recognition, where sufficiently high precision 3D depth data is required, and are oriented towards a user's face as they are looking at the device's display. It may be desirable, however, to have a system that can capture high resolution 3D data from a surgical scene while providing visual guidance to a surgeon on the device's display.

The embodiments further include provisions that enable such ‘forward facing’ sensors to gather 3D depth data for an anatomical structure even as the display of the electronic device and the sensors themselves are oriented towards the surgeon. In particular, some embodiments use a reflector assembly (comprised of mirrors, prisms, and/or lenses) to redirect the path of light entering/leaving the sensors so that the sensors are able to scan anatomical structures even when the sensors are not oriented towards those same anatomical structures.

In some embodiments, rather than use a reflector assembly, registration may be performed in two phases or stages. In a first phase, a surgeon orients the electronic device so that a first set of sensors capable of detecting the highest resolution 3D depth data are oriented towards the surgical scene, including the particular anatomical structure of interest. At this point, the first set of sensors gather 3D data and register the electronic device to the anatomical structure. After this initial registration, during a second phase, the surgeon orients the electronic device so that the display and the first set of sensors are oriented towards the surgeon and away from the anatomical structure. At this point, data may be gathered using a second set of sensors (or optionally, a singular sensor) disposed on the opposite side of the electronic device from the first set of sensors, and which are now oriented towards the surgical scene/anatomical structure. This second set of sensors, which may include a Light Detection and Ranging (LIDAR) sensor, may have less accuracy in detecting depth and thereby provide coarser three-dimensional models for registration than the data provided by the first set of sensors. However, the data gathered by the second set of sensors may still be sufficiently accurate to maintain registration with the anatomical structure once the initial registration has been performed and a sufficiently accurate rendering of the anatomical structure has been scanned and stored in memory.

In some embodiments, an auxiliary sensor system may be used. The auxiliary sensor system may be separate from the electronic device and may communicate with the electronic device (for example, wirelessly). The auxiliary sensor system may be used to provide high resolution 3D depth data for registering anatomical structures. In some cases, the attachment apparatus is configured to receive the auxiliary sensor system. The auxiliary sensor system is positioned within the attachment apparatus such that it faces out towards the anatomical structure while the surgeon is viewing the display of the electronic device.

In some embodiments, rather than use a reflector assembly, the exemplary system may utilize a display on a second display device that is separate from the electronic alignment device. In some embodiments, visual information for guiding a surgeon to place a medical device at a selected entry point and/or for guiding the surgeon in aligning the medical device as the medical device is inserted into the target area may be sent to, and displayed by, a secondary display device. This allows the surgeon to view guidance information even as the surgeon holds the electronic device with the first set of sensors detecting the 3D depth data oriented towards the patient (in which case the display of the electronic device is facing away from the surgeon).

The electronic device could be any suitable device with one or more sensors for performing registration and determining an orientation of the medical alignment device. The electronic device may be capable of running software required for performing one or more functions, as discussed below. Exemplary electronic devices could comprise any mobile computing device. In some embodiments, an electronic device may comprise a smartphone, such as an iPhone, a Windows phone or an Android phone. In some embodiments, an electronic device could comprise a tablet, such as an iPad, an Android tablet, or any other suitable computing tablet. In other embodiments, an electronic device could include a remote control with an orientation sensor and camera, such as a video game controller.

As used herein, the term “medical device” may refer to any instrument, tool, apparatus, implement, machine, contrivance, implant, in vitro reagent, or other similar or related article, including a component part, or accessory which is intended for use in the diagnosis of disease or other conditions, or in the cure, mitigation, treatment, or prevention of disease, in humans or other animals, or intended to affect the structure or any function of the body of humans or other animals. Medical devices may include, but are not limited to, surgical instruments, diagnostic equipment, implants, prosthetics, drug delivery systems, imaging systems, monitoring devices, and therapeutic equipment. Medical devices may include surgical instruments (or tools) and surgical hardware devices. In some cases, surgical instruments are used to insert/implant surgical hardware devices. As one example, an insertion instrument may be used to insert a guide pin (hardware) into a glenoid during shoulder surgery. In another embodiment, a surgical instrument may be a drill for forming holes in bone. In another embodiment, a surgical instrument may be a reamer. Examples of surgical hardware may include, but are not limited to guide pins, screws, cages, and other implants.

The term “module” may be used in reference to computational software, computational hardware and/or combinations of software and hardware. For example, a software module may comprise any suitable algorithms for accomplishing a desired function. As discussed below, software modules may be used to calculate and plan surgical trajectories, register different components to a common reference frame, determine the orientation of an electronic device and an associated medical device relative to a selected reference frame, provide an augmented reality user interface, as well as perform other suitable functionality.

For convenience, various terms may be used to describe planes of a body, such as the body of a patient undergoing a surgical procedure. The sagittal or median plane, as used herein, refers to a plane dividing a body into left and right sides. The coronal or frontal plane refers to a plane dividing the body into forward and rearward sides. The transverse plane refers to a plane dividing the body into an upper portion and a lower portion.

The terms “trajectory”, “orientation” and “angle” are also used to describe positions of various components. It may be appreciated that a trajectory or orientation may be determined relative to any suitable reference frame, and in some cases, relative to one or more of a sagittal plane, a coronal plane, or a transverse plane of a body, such as a patient's body. Likewise, an angle may be determined relative to any suitable reference frame, and in some cases, an angle may be determined relative to one or more of the sagittal plane, the coronal plane, or the transverse plane. A sagittal angle, for example, is an angle relative to a sagittal plane. A coronal angle is an angle relative to a coronal plane. A transverse angle is an angle relative to a coronal plane. In some cases, a three-dimensional insertion angle for a component (such as a medical tool) is determined and may have angular components relative to each of the sagittal, coronal, and transverse planes.

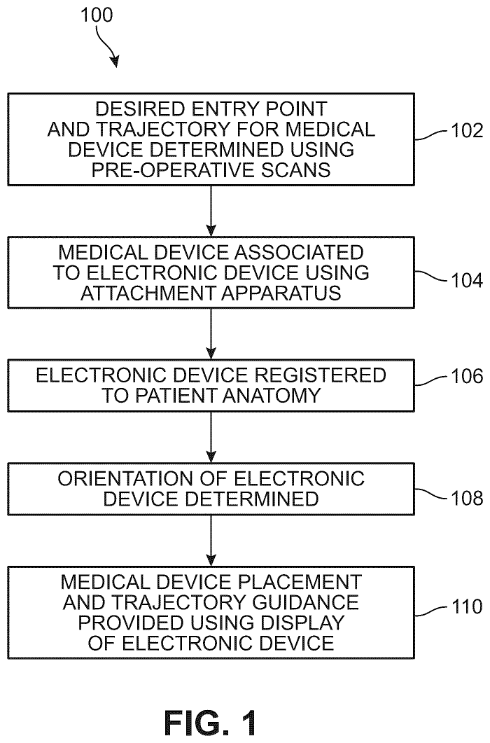

FIG. 1 is a schematic view of a process 100 for using a medical alignment system to guide the placement (entry point) and trajectory (orientation) of a medical device inserted into a bone or other tissue at a surgical site. The particular medical device used, as well as the bone/tissue where the device is inserted, may depend on the type of surgery being performed. For example, in a spinal fusion procedure, the medical device may be a drill, probe, or screw to be inserted into a pedicle. As another example, in a shoulder replacement procedure, the medical device may be a guide pin, reamer, drill bit, or other tool used to hold/insert another medical device.

Starting in operation 102, a surgeon or other user may determine a desired entry point and trajectory for a medical device (such as a guide pin) using pre-operative scans. For example, a surgeon may capture diagnostic images of a patient's anatomy using X-rays, fluoroscopic imaging, or other using other diagnostic imaging techniques. Based on these images, the surgeon may select a designated entry point and trajectory for a medical device. As one example, for a spinal fusion procedure, a surgeon may select a desired entry point and trajectory for a screw. As another example, for a shoulder replacement procedure, a surgeon may select a desired entry point and trajectory for a guide pin.

In some embodiments, as discussed below, the selection of an entry point and trajectory may be facilitated using an electronic alignment device. The electronic alignment device may include software that allows a surgeon to select a designated entry point and/or trajectory using, for example, a user interface of the device.

For clarity, the term “designated trajectory” or “designated orientation” is used to refer to the desired or intended orientation of a medical device (for example, an instrument or surgical hardware) for a surgical procedure. Because the orientation of a component may be measured using one or more angles relative to one or more reference planes (such as the sagittal, coronal, and/or transverse planes), the designated orientation may also be referred to as a “designated insertion angle”, or designated alignment angle, which may be a three-dimensional angle in some cases.

In some cases, diagnostic images are captured in particular reference planes of the patient's body. For example, diagnostic images may be captured along the sagittal, coronal, and/or traverse planes of the body. In some cases, surgical planning software allows a surgeon to designate a desired insertion angle along one or more of these planes such that an overall 3D insertion angle/orientation may be determined by the software. As discussed below, this designated orientation may then be transformed to an orientation within a different reference frame, for example, a reference frame determined by registering an electronic device with an anatomical structure of the patient.

In some cases, the pre-operative scans are taken such that the designated trajectory (orientation) may be determined relative to gravity. This may allow the electronic device to convert the designated trajectory from a reference frame associated with the pre-operative scans to a reference frame determined by gravity. The designated entry point and trajectory may then be stored in memory by the system and used during the surgical procedure to help guide the surgeon in inserting a medical device at the desired location and with the desired trajectory, as discussed in further detail below.

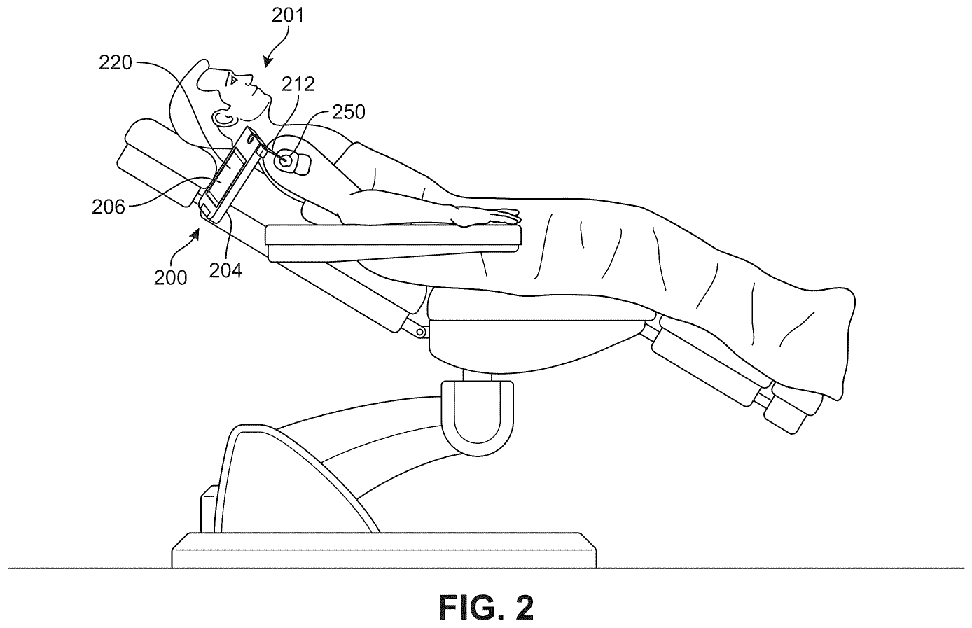

In operation 104, the medical device may be associated to the electronic device using an attachment apparatus. Specifically, the electronic device may be placed within the attachment apparatus, and the medical device may be separately coupled to the medical device. This results in a configuration in which the orientation of the medical device is fixed with respect to the electronic device such that knowing the orientation of the electronic device allows the system to calculate the orientation of the medical device. In some cases, to calculate the orientation of the medical device the system accounts for a fixed angular relationship between the electronic device and the medical device and performs the associated angular transformation (for example, using suitable rotational matrices). As an example, as seen in FIG. 2, a medical device 212 may have a perpendicular orientation with respect to an electronic device 206 when both are connected to an apparatus 204.

In operation 106, the electronic device may be registered to the patient anatomy. This process of registration may create a common frame of reference. This common frame of reference allows the system to compare relative locations and orientations between anatomical structures of the patient, the electronic device itself, and the designated entry points and trajectories determined during the pre-operative stage in operation 102. As an example, in a shoulder surgery procedure, the electronic device may be registered to the anatomy of the shoulder, and specifically to the glenoid which has an identifiable geometry. This allows the electronic device to transform entry points and/or orientations between a reference frame associated with the anatomical structure and a reference frame used in determining the designated entry point and trajectory from diagnostic images. The device may also transform between any of these reference frames and a reference frame determined by gravity, and/or any other suitable reference frame.

In operation 108, the orientation of the electronic device may be determined relative to a suitable reference frame. As discussed in further detail below, this may be done using a variety of different methods, including using LIDAR data, gyroscopic data, accelerometer data, or other suitable data.

In operation 110, the electronic device may be used to provide placement and trajectory guidance to a surgeon for positioning and aligning the medical device. In some cases, this guidance may be provided by superimposing an image of a virtual medical device over a real-time image of the patient's anatomy (such as superimposing a virtual guide pin over a live image of the glenoid). The virtual medical device may have a designated entry point and orientation with respect to the real-time image of the anatomy. As part of the process, software running on the electronic device may convert entry point and trajectory information selected by the surgeon during the pre-operative phase (as in operation 102), which is associated with a reference frame determined by the orientations of the pre-operative scans, into transformed entry point and trajectory information that is associated with the reference frame created during registration of the electronic device with the patient's anatomy.

It may be appreciated that the exemplary systems and methods described herein may be applied in a variety of different surgical contexts, including use in various orthopedic procedures. For example, the exemplary systems and methods may be useful in any situations where a surgeon requires guidance in placing and aligning any type of medical device during a procedure.

The exemplary embodiments may use any of the systems and methods for designating entry point and trajectory information, as well as any of the systems and methods for determining the orientation of an electronic device and attached medical device that are disclosed in U.S. Pat. No. 11,000,335, to Dorman, issued on May 11, 2021, and titled “System and Method for Medical Device Placement in Bone,” the entirety of which is herein incorporated by reference, and in U.S. Pat. No. ______, to Dorman, currently U.S. Patent Publication Number 2023/0131831, filed Oct. 20, 2022, and titled “Attachment Apparatus to Secure a Medical Alignment Device to Align a Tool,” the entirety of which is herein incorporated by reference.

One useful application for an alignment system is in the context of total shoulder arthroplasty (TSA), which is a surgical procedure used to treat severe shoulder joint conditions, such as osteoarthritis, rheumatoid arthritis, and post-traumatic arthritis. The procedure involves replacing the damaged or diseased parts of the shoulder joint with artificial components, typically including a metal ball attached to a stem and a plastic socket. While TSA has been successful in reducing pain and improving function for many patients, there is an ongoing need to develop better surgical techniques to enhance outcomes and reduce complications.

An important step in performing total shoulder arthroplasty is the accurate placement of a guide pin. Accurate placement of the guide pin may be important for properly positioning the glenoid component. In some aspects, the guide pin is inserted into the glenoid using a guide that corresponds to the shoulder configuration. The guide pin may then remain in place, allowing cannulated instruments to be used to prepare the glenoid for the prosthetic glenoid or glenoid baseplate.

To place the guide pin, a surgeon must know both where to place the pin (the entry point) and the orientation of the guide pin as it is inserted (the trajectory). A surgeon may plan the entry point and trajectory for the guide pin prior to the procedure. Because the anatomy of the shoulder is not generally fixed during a surgical procedure, it may be difficult to reproduce the exact entry point and trajectory during surgery. Therefore, an exemplary application of the alignment system of the embodiments is in helping a surgeon first identify a desired entry point and trajectory for the guide pin prior to surgery, and then guiding the surgeon in achieving the desired entry point and trajectory for the guide pin during the surgical procedure.

FIG. 2 is a schematic view of a patient 201 undergoing a shoulder replacement procedure. As shown in FIG. 2, an alignment system 200 comprises an attachment apparatus 204 and an electronic device 206. Alignment system 200 may be further coupled to a medical device 212. In this example, medical device 212 may comprise a guide pin. Using alignment system 200, a display 220 of electronic device 206 may provide guidance for aligning medical device 212 with a designated entry point and trajectory on the glenoid 250 that have been determined during the pre-operative phase.

For purposes of illustration, medical device 212 is shown in isolation without any other attachment components. It may be appreciated, however, that in surgery medical device 212 may be further attached to another tool for placing, drilling, or otherwise inserting medical device 212 into glenoid 250. In some cases, medical device 212, which may be a guide pin, is attached at one end to a surgical drill. This allows the surgeon to drill the guide pin into glenoid 250 once the target entry point and trajectory have been realized.

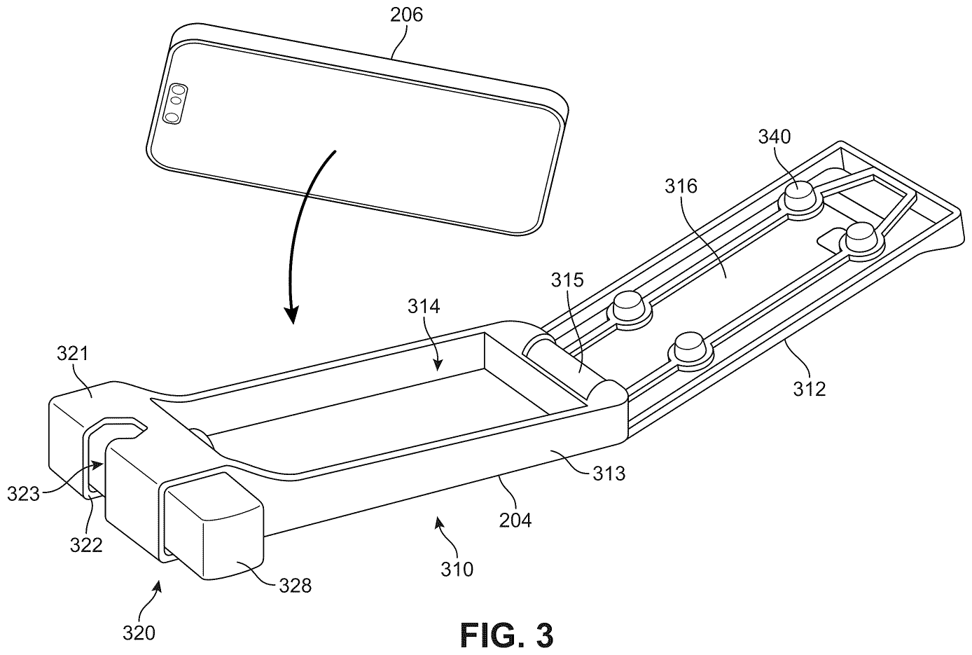

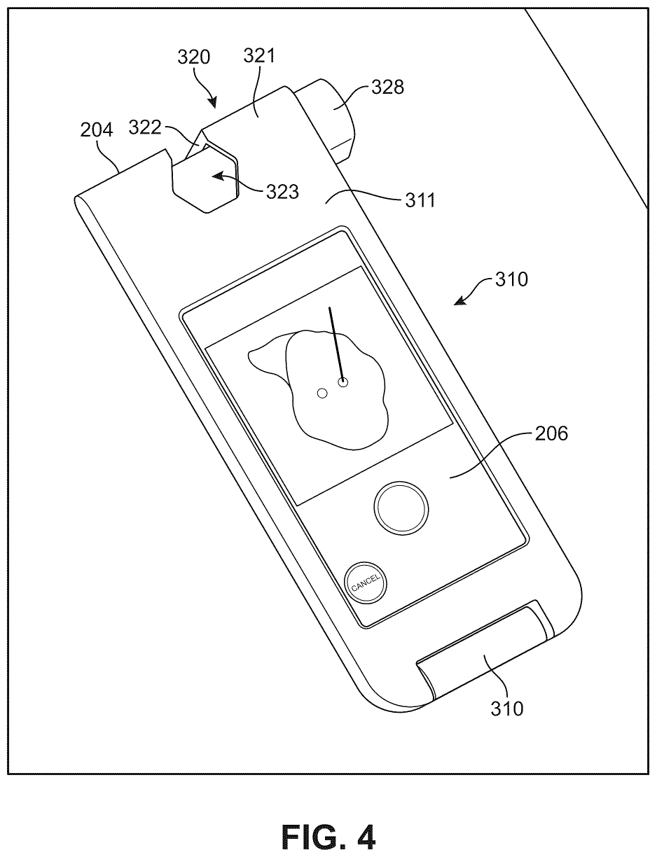

FIGS. 3 and 4 are schematic views showing components of the exemplary alignment system. In FIG. 3, electronic device 206 may be inserted into attachment apparatus 204. In FIG. 4, electronic device 206 has been enclosed within attachment apparatus 204.

As shown in FIGS. 3 and 4, attachment apparatus 204 (“apparatus 204”) comprises a first portion 310 to secure electronic device 206 such that electronic device 206 does not move relative to a medical device when the medical device is attached to apparatus 204. First portion 310 may be configured as a case-like structure to hold electronic device 206 in a secure manner. First portion 310 may be comprised of a first (front) wall 311, a second (rear) wall 312, and a set of peripheral walls 313. These walls further bound an interior cavity 314 (see FIG. 3) that is sized and shaped to receive electronic device 206.

First portion 310 may be configured to provide a secure fit when an electronic device is inserted into apparatus 204. In some embodiments, the secure fit may be provided by a frictional force produced when the distance between two or more adjacent walls of first portion 310 are less than a corresponding dimension of the electronic device. This dimensional difference slightly compresses or holds the electronic device and slightly bends first portion 310, thus creating a clamping force that results in the frictional force. First portion 310 may be manufactured to accommodate various sized electronic devices. For example, the electronic device may be an iPod™, a phone (e.g., iPhone™ 14, iPhone™ 10, etc.), a tablet (e.g., iPad™), a smart watch (e.g., Apple Watch™ Series 8, Apple Watch™ Series 5), or any other electronic device configured for these purposes. As such, apparatus 204 may be available in multiple sizes that are sized and shaped to fit the respective electronic device. It should also be understood that an apparatus may accommodate an electronic device that includes a case.

First portion 310 may further include a hinge 315 that couples to a door 316 of rear wall 312. When door 316 is open, electronic device 206 may be inserted into interior cavity 314. Once electronic device 206 is placed into the opening, door 316 may rotate about hinge 315 to mate with corresponding peripheral walls 313 so as to enclose and further secure electronic device 206. In some embodiments, door 316 may be locked into place via a snap lock (not shown).

In some embodiments, the secure fit between apparatus 204 and electronic device 206 may be facilitated by one or more protrusions 340 that are attached to an inner side of door 316. For example, the protrusions may comprise a tab (e.g., foam, sponge, rubber, etc.), a spring, or other suitable component that is operable to embrace the medical alignment device.

Apparatus 204 may also comprise a second portion 320 that may be configured to couple to a portion of a medical device, including instruments, surgical hardware, or other components. Second portion 320 is comprised of a set of exterior walls 321 that may interface with a medical device. To this end, second portion 320 may include an inner alignment surface 322 for coupling to a medical device. In some embodiments, inner alignment surface 322 may have a generally partial cylindrical profile around an axis corresponding to a thickness of apparatus 204. Inner alignment surface 322 defines an opening 323 configured to receive the medical device such that a cylindrical portion of the medical device is engageable or mateable to second portion 320. In some cases, opening 323, defined by inner alignment surface 322, has a hexagonal shape. When second portion 320 is coupled to or positioned adjacent to the medical device, the longitudinal axis of the instrument is aligned with the axis of inner alignment surface 322 to provide proper alignment. Moreover, when attached to second portion 320 using this coupling, the relative orientation of the medical device is fixed with respect to any particular axis of second portion 320.

Second portion 320 may further include a holding mechanism. The holding mechanism comprises a holding member 328 that is inserted through a channel within second portion 320 so as to open and close access to opening 323. Moreover, the holding mechanism may be biased in a closed position (for example, using a spring-biased plunger), so that a user is required to press an end of holding member 328 inwards (into second portion 320) so that the holding mechanism does not block access to opening 323.

In other embodiments, second portion 320 may comprise other mechanisms for holding a medical device. For example, in some cases, second portion 320 may comprise an opening with a diameter that fits with the diameter of some portion of a medical device such that the device is held in place by friction within the opening. In some cases, the opening may be filled with a material that facilitates the frictional fit between a portion of a medical device and the opening within second portion 320. In some embodiments, second portion 320 may include other types of holding mechanisms for securing a medical device. For example, the holding mechanism may comprise a clamp or vise-like structure that can be tightened around the medical device. In some cases, the holding mechanism may utilize a set of adjustable jaws that can be opened and closed to accommodate medical devices of different sizes and shapes.

In other embodiments, the holding mechanism may employ a magnetic coupling system. This may include one or more magnets integrated into second portion 320 that attract and hold ferromagnetic portions of the medical device. The magnetic coupling may provide a secure hold while allowing for easy attachment and detachment of the medical device. Some embodiments may utilize a quick-release mechanism, such as a spring-loaded pin or ball detent system. This type of mechanism may allow for rapid engagement and disengagement of the medical device, which may be beneficial in time-sensitive surgical procedures. In certain cases, the holding mechanism may incorporate a threaded coupling system. This may involve a threaded receptacle in second portion 320 that corresponds to threads on the medical device, allowing for a secure screw-in attachment.

Apparatus 204 may also be configured with various openings, which are discussed in further detail below, allowing both user interaction with an electronic device, as well as allowing sensors of the electronic device to receive signals from the surgical environment.

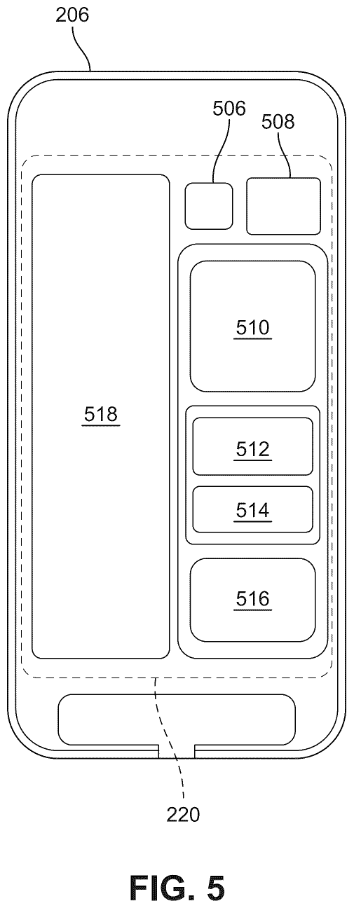

FIG. 5 is a schematic view of some of the components of electronic device 206. Electronic device 206 includes a plurality of components typically found in a cellphone or smart phone, including, as shown in FIG. 5, at least a display 220, a global positioning system (GPS) sensor 506, a processor 510, a communication module 512 (e.g., for enabling various wireless network communications, such as via code division multiple access (CDMA) and/or global system for mobile communications (GSM) networks, as well as one or more of Bluetooth, WiFi, and/or other wireless communication technologies), a memory 514, a connection module 516 (e.g., to enable wired connections, such as via universal serial bus (USB), Lightning connector, etc.), and a battery 518.

Electronic device 206 may include additional sensors for determining an orientation of the device. These sensors may include an inertial measurement unit (IMU) 508. An IMU may comprise multiple components such as accelerometers and gyroscopes. Accelerometers may measure linear acceleration along different axes, while gyroscopes may measure angular velocity around different axes. By combining data from these components, the IMU may determine the device's orientation in three-dimensional space. Additionally, the IMU may be used to track motion, calculate velocity, and estimate position changes. In some cases, the data from the IMU may be processed using sensor fusion algorithms to provide more accurate and stable orientation estimates, which may be particularly useful in medical alignment applications. In some embodiments, data from IMU 508 may be processed using a filter to estimate Euler angles for the device axes relative to a predetermined coordinate system.

Electronic device 206 may include additional sensors for capturing external information, including optical data and other kinds of data for performing imaging, ranging, and registration. In some embodiments, electronic device 206 includes one or more sensors that enable 3D depth sensing. 3D depth sensing systems may use various technologies to capture three-dimensional information about the surrounding environment. For example, some devices may employ structured light systems that project a known pattern onto a scene and analyze the deformation of that pattern to calculate depth. Other devices may use time-of-flight (ToF) sensors that measure the time it takes for light to travel to an object and back to determine distance. Stereo vision systems, which use two or more cameras to capture images from slightly different angles and calculate depth through triangulation, may also be utilized. In some cases, these 3D depth sensing systems may be combined with other sensors, such as RGB cameras or infrared sensors, to provide more comprehensive spatial information. The data from these systems may be used for various applications, including augmented reality experiences, improved facial recognition, and enhanced photography features. In one embodiment, electronic device 206 may utilize TrueDepth technology, developed by Apple™.

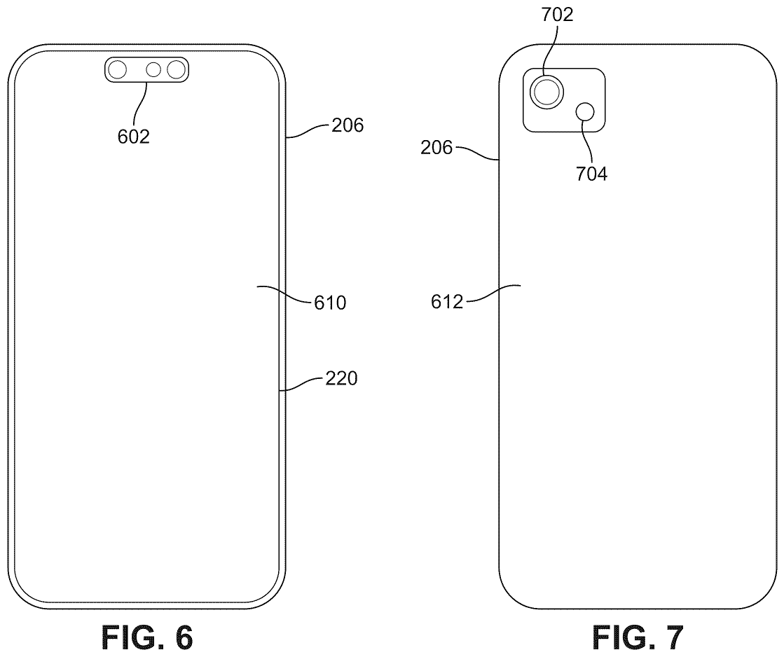

Referring now to FIG. 6, electronic device 206 may include a sensor assembly configured to enable TrueDepth or similar 3D depth sensing technologies. Specifically, electronic device 206 may include a sensor assembly 602 disposed on a first (front) side 610 of electronic device 206, which is the same side where display 220 is located. For purposes of illustration, sensor assembly 602 is shown schematically, with three sensors visible, however as described herein, sensor assembly 602 may employ more than three sensors or components, some of which are not easily visible on electronic device 206.

The sensor assembly may comprise multiple components working together to capture depth information and create a detailed 3D map of objects or scenes. In some embodiments, this assembly may include a flood illuminator that projects infrared light onto the scene, an infrared camera that captures the infrared light reflected back from objects, a dot projector that emits over 30,000 invisible infrared dots onto the scene, and a standard RGB camera for capturing color images. The flood illuminator may bathe the scene in infrared light, which is invisible to the human eye but detectable by the infrared camera. This provides a base layer of illumination for the depth sensing process. The dot projector may create a precise pattern of infrared dots on the scene. As these dots hit surfaces at different distances, they appear displaced from their original projected pattern when viewed by the infrared camera. The infrared camera may capture an image of the reflected dot pattern. By analyzing how the pattern is distorted compared to the known projected pattern, the system can calculate the depth of objects in the scene. The RGB camera may capture standard color images that can be combined with the depth information to create textured 3D models or enable augmented reality applications. In some cases, the assembly may also include additional sensors such as an ambient light sensor to adjust for different lighting conditions. In some cases, the assembly may also include a proximity sensor. The data from all these components may be processed by specialized algorithms running on the device to create a detailed 3D depth map. This depth information may be used for various purposes in medical alignment applications, such as precise registration of anatomical features or accurate measurement of distances within the surgical field.

Referring to FIG. 7, electronic device 206 may include additional sensors on a second (rear) side 612. Electronic device 206 may include an RGB camera 702 and a LIDAR sensor 704. In some cases, RGB camera 702 may be used to capture images and/or video of a surgical scene. This may allow real-time images from the surgical scene to be shown on display 220, enabling the use of augmented reality to provide guidance during a surgical procedure. In some cases, RGB camera 702 may be used to capture diagnostic image information, including allowing a surgeon to take a picture of an image generated by an X-ray system, fluoroscopy system, or other suitable diagnostic system. These captured images may then be imported into software that facilitates pre-operative planning, including software for designating an entry point and trajectory for a medical device.

In some cases, lidar sensor 704 may be used to make depth or ranging measurements. Lidar, which stands for Light Detection and Ranging, uses laser pulses to measure distances and create detailed 3D maps of the surrounding environment. In some embodiments, the lidar sensor may be used for various purposes in medical alignment applications, particularly for identifying and registering specific anatomic features. The lidar sensor may be used to capture 3D scans of a patient's anatomy. This may allow for precise measurements of anatomical structures, which can be particularly useful in orthopedic procedures. For example, in a shoulder replacement surgery, the lidar sensor may be used to create a 3D model of the patient's glenoid, scapula, or other structure. In some cases, the lidar sensor may be used to identify specific anatomical landmarks. The system may analyze the 3D point cloud data generated by the lidar sensor to detect edges, curves, and other distinctive features that correspond to known anatomical structures. This capability may be particularly useful for registering the system to the patient's anatomy.

The lidar sensor may also be used in conjunction with other sensors on the electronic device to improve the accuracy of registration. For instance, data from the lidar sensor may be combined with images from the RGB camera to create a more comprehensive representation of the surgical site. This fusion of data may enhance the system's ability to identify and track specific anatomical features throughout the procedure.

In some embodiments, the lidar sensor may also be used to determine the orientation of the electronic device. The lidar sensor may capture detailed 3D scans of the surrounding environment, including fixed objects or surfaces in the surgical room. By analyzing the spatial relationships between these fixed points and comparing them to previous scans, the system may calculate changes in the device's position and orientation. For example, the lidar sensor may detect the edges of surgical equipment, walls, or other stationary objects in the room. As the electronic device moves, the relative positions of these fixed points in the lidar scans may change. By tracking these changes, the system may determine how the device has rotated or translated in space.

In some cases, the lidar-based orientation determination may be used in conjunction with data from the IMU for more robust and accurate orientation tracking. The lidar data may help correct for drift in the IMU measurements over time, providing a more stable reference frame for the device's orientation.

Although both the sensor assembly 602 on first side 610 of device 206 and LIDAR sensor 704 on second side 612 of device 206 may be used to create 3D maps of a patient's anatomy, there may be distinctions in the type of resolution/precision provided by these different types of sensor systems. In particular, in some cases, sensor assembly 602, which may use structured light to map anatomical structures in some cases, may provide more precise maps of a patient's anatomy than that provided solely using LIDAR sensors. In particular, to obtain the sort of precision needed to identify the precise orientation of an anatomical structure, such as the glenoid in the shoulder, data provided solely by lidar sensor 704 may not be sufficient in some instances and so data from sensor assembly 602 may be required.

In cases where sensor assembly 602 uses structured light technology to obtain 3D depth data, the data provided by sensor assembly 602 to software running on electronic device 206 may be referred to as ‘structured light data’. By contrast, data provided by LIDAR sensor 704 may be referred to as ‘LIDAR data’. Using structured light data may allow the system to build more accurate/precise 3D maps of anatomical structures, and thereby provide more accuracy in registering the electronic device to the anatomical structure compared with using only LIDAR data. As discussed below, in some cases 3D depth data with a higher resolution, such as structured light data, may be used for some phases of registration, while LIDAR data, which provides less resolution than structured light data, may be used for other phases of registration.

Many devices, such as cell phones, employ a sensor assembly capable of more precise 3D depth sensing on a front of a device, where the display is located, as this is most suitable for many smart-phone applications including facial recognition. To facilitate using an electronic device to guide a surgeon in positioning a medical device, the embodiments utilize an attachment apparatus that allows sensory assembly 602 to receive information from the surgical setting even while the surgeon is observing the display (in which case sensory assembly 602 is facing towards the surgeon). This is accomplished using a configuration of openings within the apparatus as well as a reflector assembly (and optional lenses) that allows light to be guided between a surgical setting associated with a rear side of the electronic device and sensory assembly 602 associated with a front side of the device. In particular, light may be conveyed between the surgical scene and sensory assembly 602 even as the electronic device is positioned with sensory assembly 602 facing away from the surgical scene.

As used herein, a reflector assembly refers to any configuration of reflectors, including both mirrors and prisms, which allows the direction of light to be changed as it passes from one location to another. In some cases, lenses may be used in combination with a reflector assembly to refocus light at one or more locations along its path.

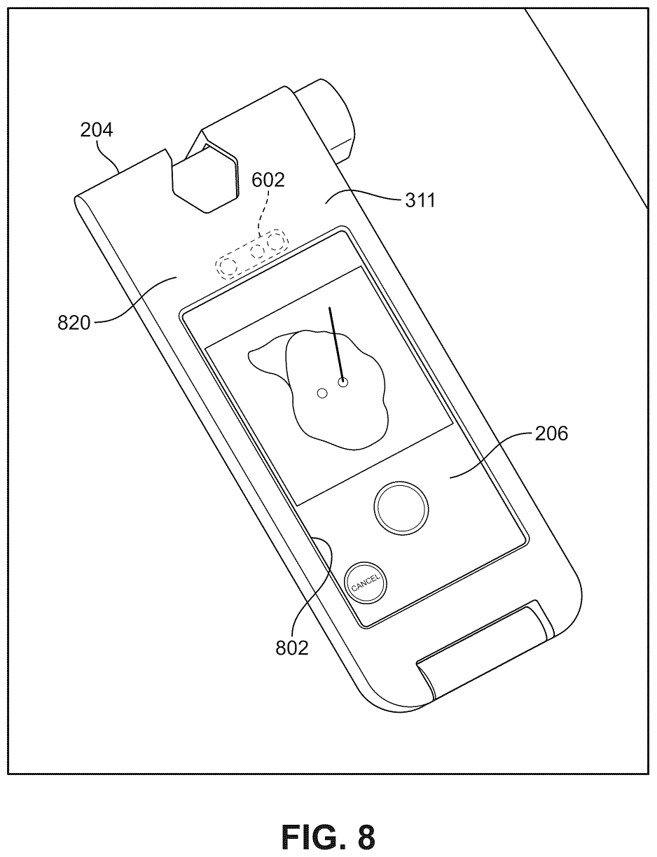

FIG. 8 shows a schematic perspective view of the first (front) side of apparatus 204 while electronic device 206 is disposed within apparatus 204. In this configuration, display 220 of electronic device 206 is visible through a display opening 802 of apparatus 204, which is located on a front side of apparatus 204. Specifically, opening 802 is disposed in first wall 311 of apparatus 204. Display opening 802 is sized and shaped so that a surgeon may view and/or touch a portion of display 220. However, as seen in FIG. 8, sensor assembly 602 (shown in phantom), which is disposed on the first (front) side 610 of electronic device 206, is covered by an upper perimeter portion 820 of apparatus 204 that bounds display opening 802 at the top.

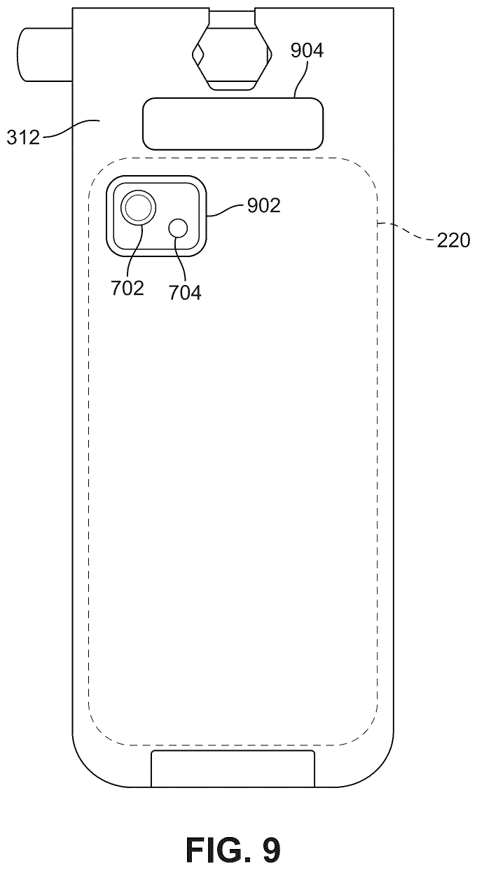

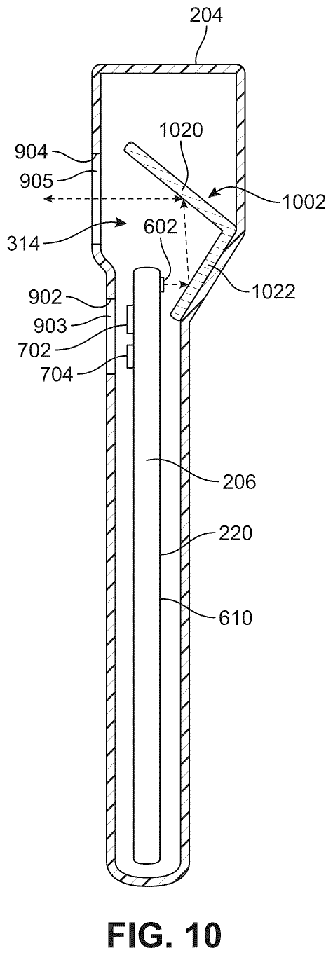

Referring now to FIG. 9, the rear side of apparatus 204 includes a first rear opening 902 in second wall 312 that allows for the use of RGB camera 702 and LIDAR sensor 704 of electronic device 206. That is, light may enter first rear opening 902 and be captured by camera 702 and/or LIDAR sensor 704. Likewise, light may be transmitted by LIDAR sensor 704 through first rear opening 902. Optionally, in some embodiments, first rear opening 902 may include a lens 903 (see FIG. 10).

Apparatus 204 may also include a second rear opening 904 on the rear side and within second wall 312. Second rear opening 904 provides an opening for light to enter and exit an interior cavity 314 of apparatus 204. Optionally, in some embodiments, second rear opening 904 may include a lens 905 (see FIG. 10).

In some embodiments, an attachment apparatus includes provisions for redirecting light entering through second rear opening 904 to an internal region of the apparatus that is associated with a sensor of the electronic device.

Referring now to FIG. 10, which shows a side schematic cross-sectional view of alignment system 200, attachment apparatus 204 may include a reflector assembly 1002. Specifically, reflector assembly 1002, comprising one or more mirrors and/or prisms, may be disposed within an interior of apparatus 204. Reflector assembly 1002 is configured so that light entering second rear opening 904 is guided into interior cavity 314 and reflects off reflector assembly 1002 to sensor assembly 602 on the front side 610 of electronic device 206. Likewise, light transmitted by one or more components of sensor assembly 602 is reflected off of reflector assembly 1002 and exits interior cavity 314 through second rear opening 904.

In the exemplary embodiment shown in FIG. 10, reflector assembly 1002 uses a first mirror 1020 and a second mirror 1022 to reverse the direction of light passing between second rear opening 904 and sensory assembly 602. This enables light captured through second rear opening 904 of the rear side of apparatus 204 to be captured by sensor assembly 602, which is oriented towards the front side of apparatus 204. Likewise, this configuration enables light transmitted by one or more components of sensor assembly 602 oriented towards the front side of apparatus 204 to be projected through second rear opening 904 of the rear side of apparatus 204. In some cases, optional lenses, such as lens 903 and lens 905, may be used to focus light.

While the exemplary embodiments use a configuration with two mirrors and a single lens, in other embodiments, any other suitable configuration of mirrors and lenses may be used. In still other embodiments, prisms may also be used to change the direction of light. For example, embodiments may include erecting prisms, amici prisms, right angle prisms, and star diagonals, which may change the direction and/or orientation of light. Still other embodiments could use a combination of mirrors and prisms. Still other embodiments could use a combination of mirrors, prisms, and lenses.

Moreover, it may be appreciated that in other embodiments the attachment apparatus may have other configurations that accommodate different arrangements of reflectors. In other embodiments, for example, portions of the attachment apparatus may extend out from the body and, using a suitable configuration of mirrors, prisms, and/or lenses, form a ‘periscope’ like structure extending from the from to the rear side of the apparatus.

In the exemplary configurations of FIGS. 8-10, second rear opening 904 may be disposed above first rear opening 902. Moreover, second rear opening 904 has a position within rear wall 312 such that second rear opening 904 is above electronic device 206, as shown in FIGS. 9 and 10. This is due to the configuration of reflector assembly 1002, which translates the path of light a vertical distance as it also reverses the direction of the light. With this particular configuration, if second rear opening 904 were disposed at the same vertical level (or below) as first rear opening 902, the presence of electronic device 206 would obscure the path of the light.

Using the exemplary configuration, sensor assembly 602 may be used to build 3D maps of a patient's anatomy even as the surgeon is viewing the display of electronic device 206. This allows the system to register the electronic device with the patient's anatomy as the surgeon is viewing information on the display. Additionally, both RGB camera information and LIDAR data of the surgical scene may be captured as sensor assembly 602 continues to receive information and facilitate ongoing registration. In some cases, images or video captured using the RGB camera may be provided as part of a real-time augmented reality experience on display 220 of electronic device 206. Likewise, as discussed below, LIDAR data may optionally be used to facilitate registration and/or to determine an orientation of the electronic device in real-time.

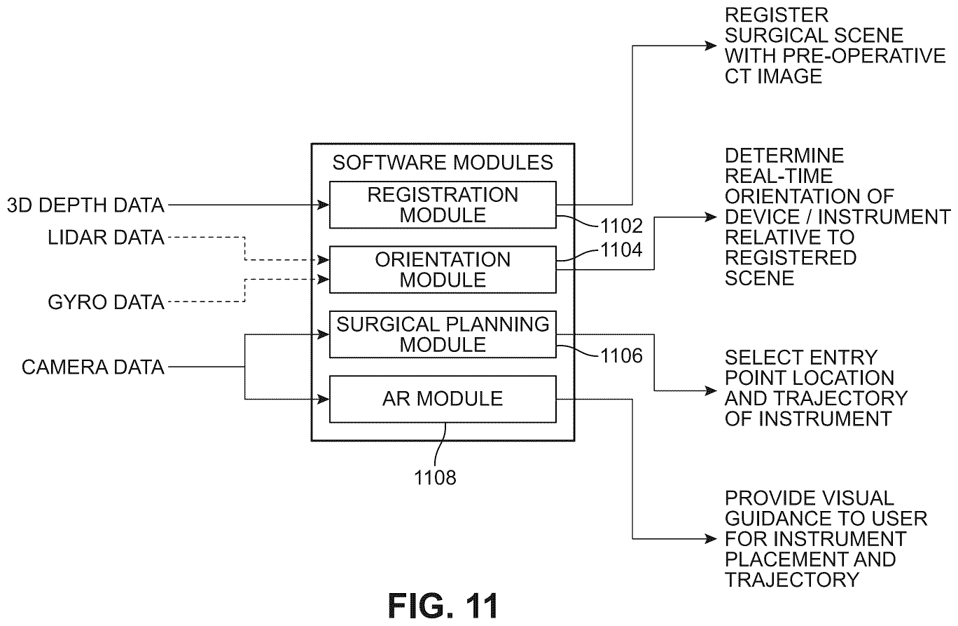

FIG. 11 is a schematic view of a set of software modules that may be stored in memory and used by electronic device 206 to facilitate various functionality. As seen in FIG. 11, the set of software modules include a registration module 1102. Registration module 1102 may include one or more algorithms for registering the surgical scene (and specifically, an anatomical structure) with a reference coordinate system. Registration module 1102 may receive 3D depth data, including structured light data. This data may be used to generate a 3D map of an anatomical structure, such as a glenoid. The 3D map may then be used to infer the relative orientation of the anatomical structure relative to a reference coordinate system. In some cases, the reference coordinate system is determined by the pre-operative images (such as CT images). As discussed, 3D depth data may be captured by sensor assembly 602 located at the front of electronic device 206.

The registration module may be configured to register a particular object in an environment and determine an associated coordinate system. In some aspects, the registration module may receive input data from various sensors of the electronic device, such as the sensor assembly, RGB camera, and lidar sensor. The module may process this data to identify and locate specific features or landmarks of the anatomical structure.

In some cases, the registration module may employ computer vision algorithms to analyze image data and detect distinctive patterns, edges, or contours that correspond to known features of the object. The module may also utilize depth information from the 3D depth sensing system to create a point cloud representation of the object and its surroundings.

Once the anatomical structure is scanned/identified, the registration module may align the detected features with a pre-existing 3D model or template of the object. This alignment process may involve techniques such as iterative closest point (ICP) algorithms or other point cloud registration methods. By matching the detected features to the known model, the module may determine the position and orientation of the anatomical structure relative to the electronic device. In some embodiments, the registration module may establish a local coordinate system based on the registered anatomical structure. This coordinate system may be defined using key landmarks or geometric features of the anatomical structure.

In the particular case of the glenoid, for example, registration may occur in part by creating a 3D model of the glenoid during a surgical procedure and using artificial intelligence or other computer visions techniques to identify features of the glenoid. This may include the identifying the general concavity of the glenoid, its distinctive circumferential shape, and dimensions such as the glenoid height, glenoid upper width, and glenoid lower width. By analyzing the concavity and other features, the system may determine the placement and orientation of the glenoid within a known reference frame, such as a reference frame associated with gravity, a reference frame associated with one of the sagittal, coronal, or traverse planes of the body, or relative to another suitable reference frame.

In some cases, the 3D model constructed by scanning the glenoid is compared to a stored model of a general glenoid. In other cases, the 3D model is compared to diagnostic images taken of the glenoid, allowing the 3D model to be registered directly to the glenoid in the diagnostic images.

The registration module may also track the anatomical structure over time, updating its position and orientation as the electronic device or the anatomical structure moves. This may involve continuously processing new sensor data and refining the registration to maintain accuracy. In some cases, the module may employ sensor fusion techniques, combining data from multiple sensors to improve the robustness and precision of the registration.

Additionally, the registration module may provide interfaces for other software components to access the registration information.

While the exemplary embodiment of FIG. 11 uses only a single data source for 3D depth information, such as from a sensor assembly capable of capturing structured light data, in other embodiments, as discussed below, registration module 1102 may also receive LIDAR data.

An orientation module 1104 may receive data from one or more sensors and may be used to determine a real-time orientation of the electronic device (and the medical device) with respect to the reference coordinate system. Moreover, because the surgical scene is registered to the reference coordinate system, the real-time orientation of the electronic device and the medical device may also be determined relative to the surgical scene.

In some embodiments, the orientation of the electronic device may be determined using LIDAR data. For example, by using LIDAR data to map out planar surfaces within the surgical scene and monitor how mapped surfaces change relative to the electronic device as it is rotated, orientation module 1104 may compute relative changes in the orientation of the electronic device. In some cases, this may be used in conjunction with data from the IMU to measure changes in orientation relative to an initial orientation, such as an initially vertical position.

In some embodiments, the orientation of the electronic device may be determined using gyroscopic data. In some cases, the orientation of the electronic device may be determined using a combination of gyroscopic and accelerometer data, which may both be provided as part of the device's IMU.

The electronic device may also include a surgical planning module 1106. This may include algorithms, as well as a user interface, which allow a surgeon to select an entry point location and trajectory for a medical device. In some cases, surgical planning module 1106 allows a surgeon to capture images (camera data) of diagnostic scans and indicate a designated entry point as well as a designated trajectory for a medical device. The surgical planning module 1106 may then store this information in memory to be retrieved and used for guiding the surgeon to align the medical device during surgery. In some cases, diagnostic images may be imported into the software without using a camera.

The electronic device may also include an augmented reality (AR) module 1108 that may be used to facilitate guiding the alignment of the medical device during surgery. In particular, AR module 1108 may provide visual guidance to the surgeon for placing and orientating the medical device in real time. In some embodiments, camera data may be fed to AR module 1108. This data may be combined with augmented reality elements indicating the desired location and orientation of the medical device, which may be generated using information from surgical planning module 1106. In some embodiments, during a surgical procedure the display of the electronic device shows a real-time feed of the surgical scene with the augmented reality elements superimposed over the scene. The surgeon is therefore intended to adjust the position of the apparatus until the medical device aligns with the augmented reality elements on the display.

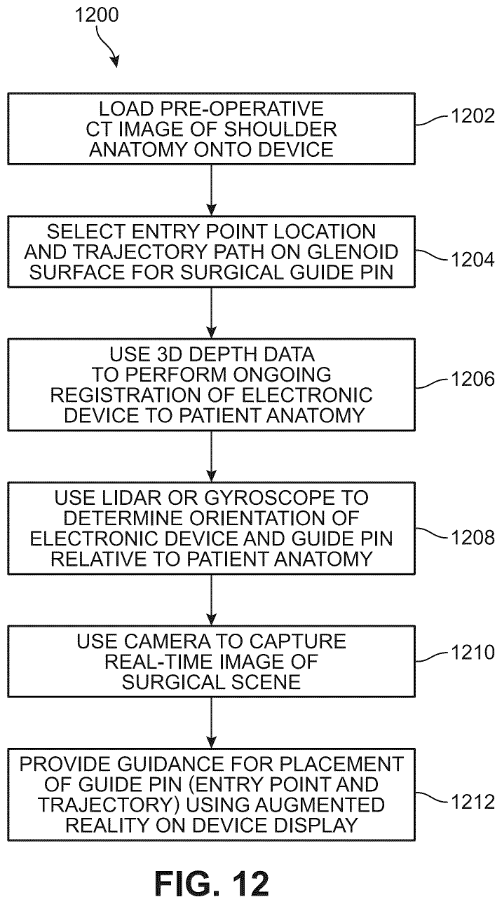

FIG. 12 is a schematic view of a process 1200 for using a medical alignment system to guide the placement (entry point) and trajectory (orientation) of a guide pin that is inserted into the glenoid during a shoulder replacement surgery. It may be appreciated that a similar procedure may be used for guiding a surgeon in finding an entry point and trajectory for other medical devices used during shoulder surgery, as well as for positioning and aligning medical devices used in any other suitable surgery.

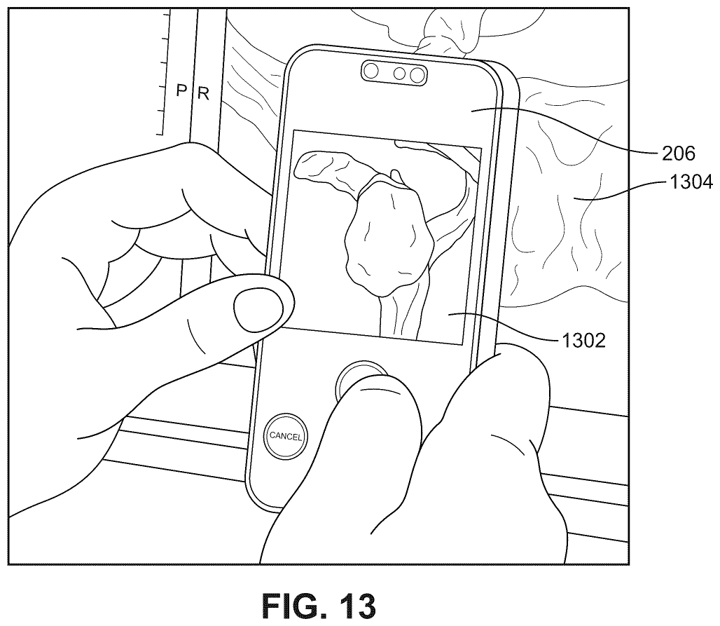

Starting in operation 1202, the surgeon may load pre-operative CT images of the shoulder anatomy onto the device. In some cases, these are diagnostic images of the patient's shoulder that may be initially captured using CT fluoroscopy. The diagnostic images may then be captured using a camera of the electronic device and loaded into the surgical planning software. An example of this operation is shown in FIG. 13, in which a surgeon uses electronic device 206 to capture an image 1302 of a diagnostic image 1304 of the glenoid.

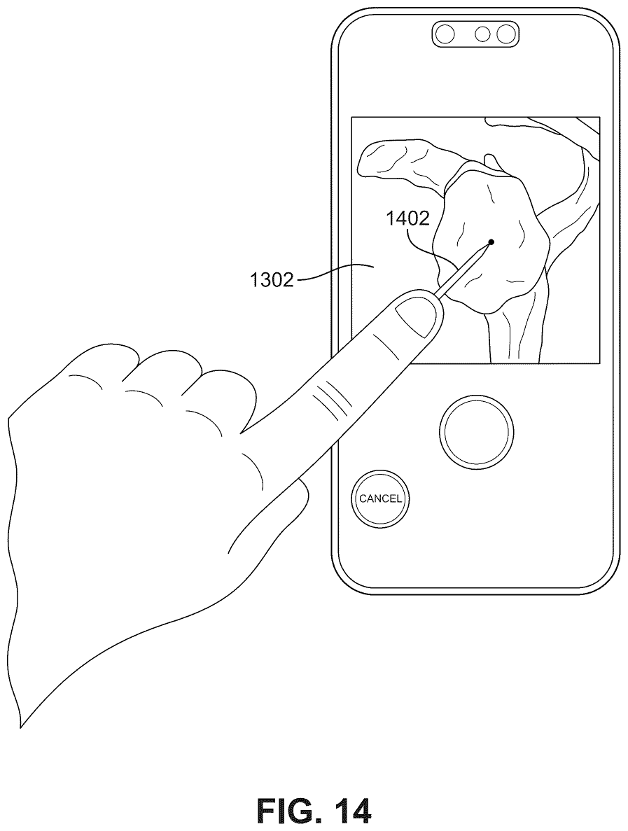

In operation 1204, the surgeon may use the surgical planning software and the acquired diagnostic images to select an entry point and trajectory (orientation) on the glenoid surface for inserting the guide pin. For example, as shown in FIG. 14, a surgeon may drag and orient a virtual guide pin 1402 over the captured image 1302 to select a desired entry point and trajectory for the guide pin into the glenoid.

In operation 1206, 3D depth data is acquired using a suitable senor assembly (such as sensory assembly 602) on the electronic device to perform initial and ongoing registration of the electronic device to the patient's anatomy.

In operation 1208, LIDAR and/or gyroscopic data (possibly combined with other data such as accelerometer data) may be used to determine an orientation of the electronic device and medical device relative to the patient anatomy. Moreover, since the electronic device has been registered with the surgical scene, the orientation of the electronic device may also be determined relative to the designated orientation of the medical device determined during the pre-operative phase.

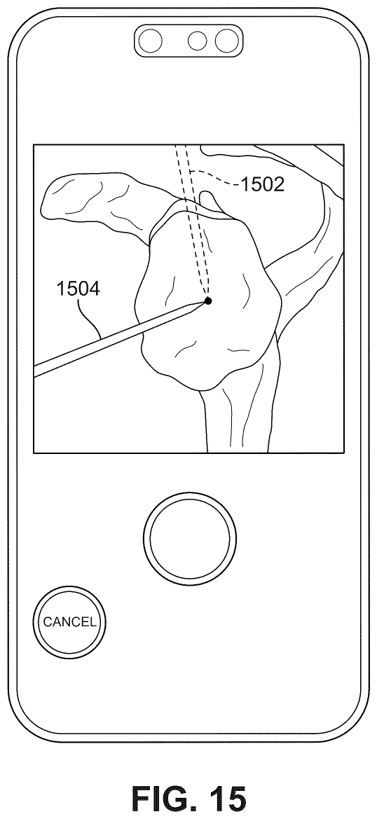

In operation 1210, a camera, such as RGB camera 702, is used to capture a real-time image of the surgical scene which may be shown on the display of the electronic device. In operation 1212, the electronic device uses augmented reality to superimpose augmented reality elements indicative of the designated entry point and orientation for the medical device over the real-time feed of the surgical scene. For example, in FIG. 15, a virtual guide pin 1502 is superimposed over the surgical scene captured by the camera so that a surgeon can visually align the physical guide pin 1504. This allows the surgeon to adjust the placement and orientation of the apparatus and electronic device until the physical guide pin 1504 has the desired entry point and orientation relative to the glenoid.

In some embodiments, both 3D depth data and LIDAR data may be used for registration. In some cases, 3D depth data, gathered, for example, using a sensor assembly on a front of an electronic device, may be used to perform an initial registration, while LIDAR data, gathered, for example, using a LIDAR sensor on a rear side of the electronic device, may be used to perform ongoing registration after the initial registration has been made. In other embodiments, 3D depth data and LIDAR data may be used simultaneously to perform registration.

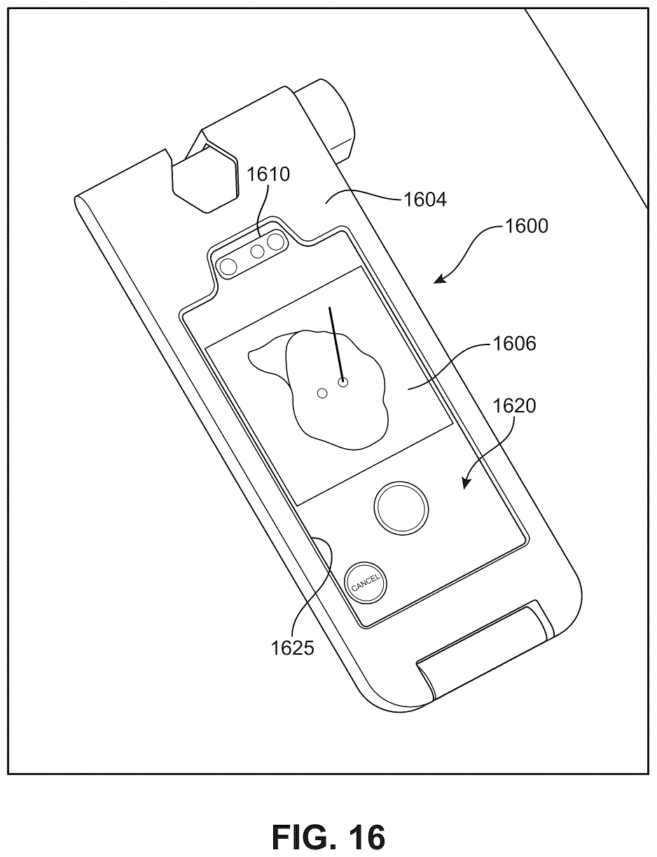

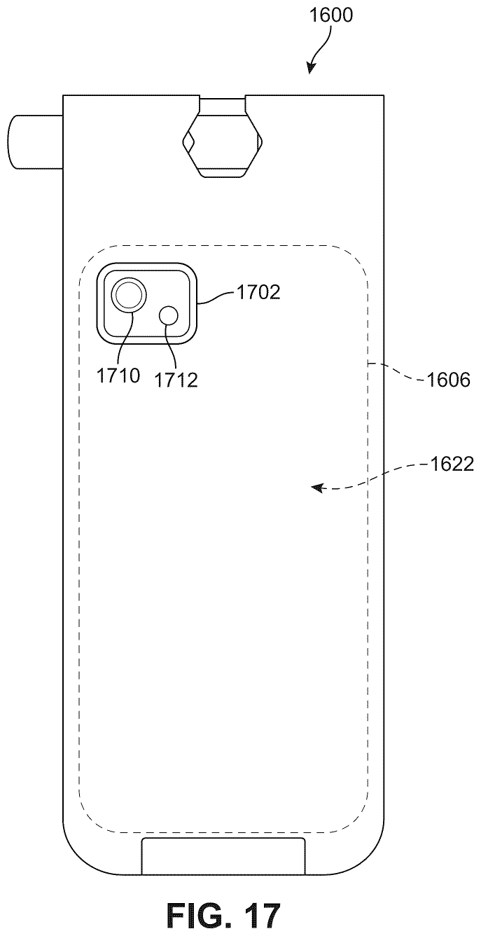

FIGS. 16 and 17 are schematic views of an embodiment of an alignment system 1600. Alignment system 1600 includes attachment apparatus 1604 and electronic device 1606. In the exemplary embodiment, electronic device 1606 includes a sensory assembly 1610 on a front side 1620 of electronic device 1606. Sensory assembly 1610 is visible through front opening 1625 of attachment apparatus 1604. Additionally, as seen in FIG. 17, apparatus 1604 includes a rear opening 1702 for an RGB camera 1710 and a LIDAR sensor 1712 disposed on a rear side 1622 of electronic device 1606.

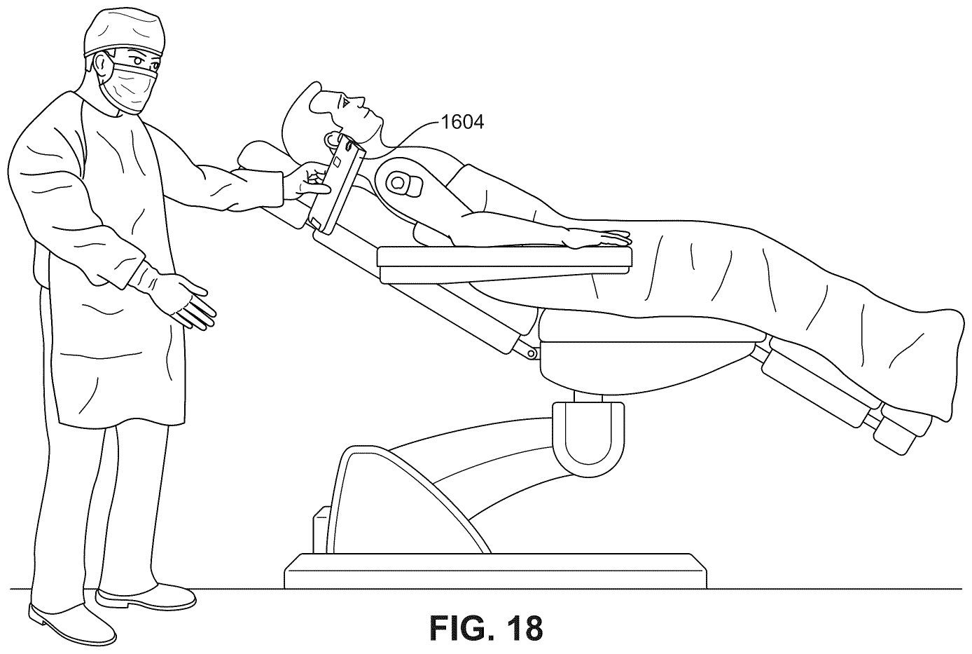

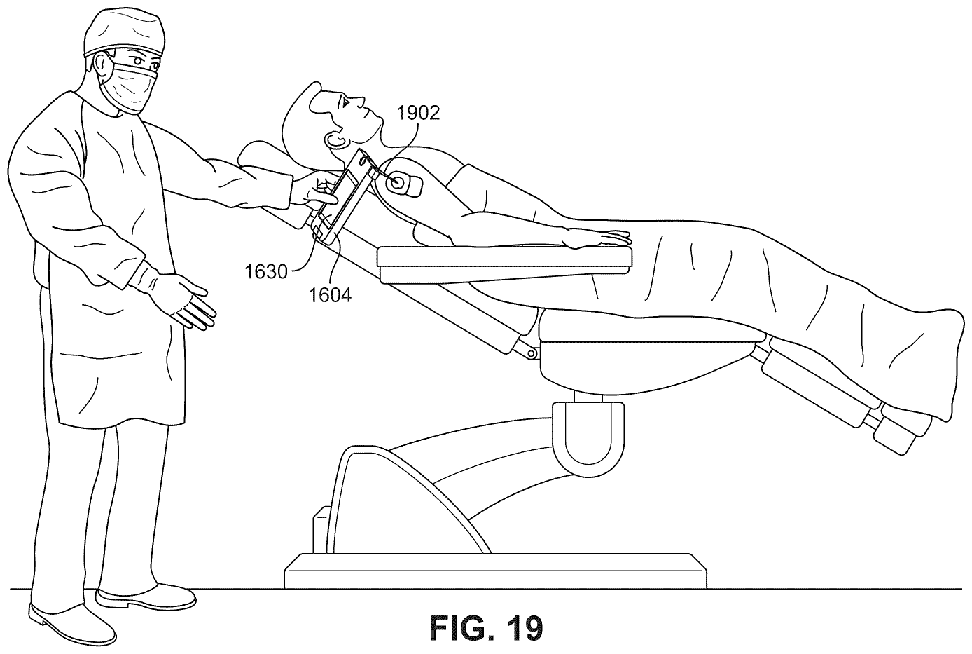

Alignment system 1600 may be operated similarly to alignment system 200 of the previous embodiment. In particular, alignment system, including software running on electronic device 1606 may include provisions for capturing pre-operative images and designating entry points and trajectories for medical devices. However, in contrast to a previous embodiment, sensor assembly 1610 on front side 1620 may only be used when the front side of electronic device 1606 is facing towards the surgical scene, rather than towards the surgeon. Therefore, the operation of registering electronic device 1606 with the patient's anatomy, such as the glenoid, is performed in two phases. During a first, or initial, phase, a surgeon may hold alignment system 1600 such that the front side 1620 of electronic device 1606 is facing towards the surgical scene, as in FIG. 18. At this time, sensory assembly 1610 is facing towards the surgical scene and may gather the necessary 3D depth data to map the patient's anatomy, such as the glenoid, and thereby register electronic device 1606 to the anatomy. Once this initial registration is complete, which may be indicated using a sound, spoken instructions, or other notification generated by electronic device 1606, a surgeon may rotate the alignment system 1600 until the rear side 1622 of the electronic device 1606 is oriented towards the surgical scene, as in FIG. 19. At this point, the display 1630 of electronic device 1606 may be visible to the surgeon. At this same time, the surgeon may also attach a medical device 1902 that is to be aligned and/or inserted to attachment apparatus 1604. Alternatively, the medical device may be attached to attachment apparatus 1604 prior to the initial registration.

With the alignment system 1600 positioned so that rear side 1622 is oriented towards the surgical scene, a live feed of the patient's anatomy, such as the glenoid, may be shown on display 1630. Additionally, augmented reality elements may be superimposed over the live feed of the anatomy to give the surgeon a visual indicator for aligning medical device 1902. Because the relative position and orientation of the patient's anatomy (such as the glenoid) may move during the procedure, registration between electronic device 1606 and the anatomy may be performed throughout the process during a second phase of registration. In this second phase, registration may be performed using LIDAR data captured by the LIDAR sensor that is oriented towards the surgical scene while display 1630 is oriented towards the surgeon.

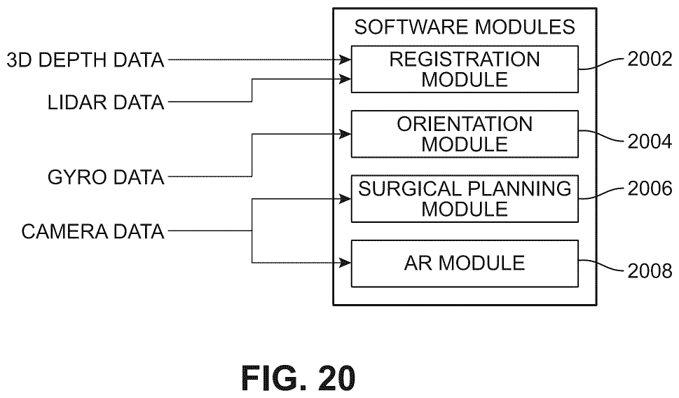

The particular use of sensor data for the exemplary alignment system 1600 is shown in FIG. 20. In this case, both 3D depth data and LIDAR data are used to register the electronic device with the patient's anatomy. That is, both 3D depth data, including possibly structured light data, and also LIDAR data are fed into registration module 2002. Additionally, gyroscopic data (and possibly accelerometer data) may fed into orientation module 2004 and used to determine the electronic device's orientation relative to the reference frame associated with the registration. Additionally, RGB camera data may be provided as inputs for both surgical planning (module 2006) and the augmented reality-based guidance for the surgeon (module 2008).

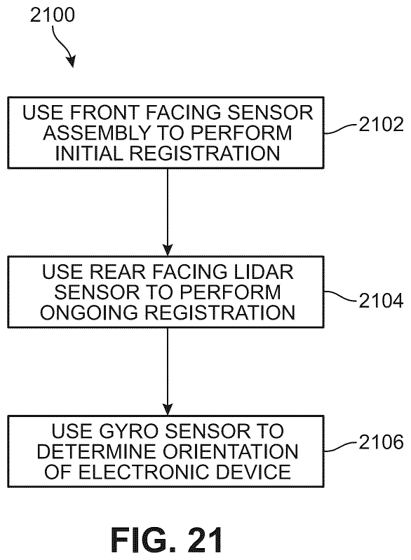

FIG. 21 is a schematic view of a process 2100 for using the exemplary alignment system of FIGS. 16-20 to provide guidance for the entry point and orientation of a medical device. In an operation 2102, 3D depth data from the front facing sensory assembly is used to perform an initial registration between the electronic device and the patient's anatomy. This registration allows the designated entry point and trajectory information to be mapped to a reference frame associated with the patient anatomy. In operation 2104, data from the rear facing LIDAR sensor is used to perform ongoing registration, once the surgeon has rotated the alignment system such that the surgeon can view the display of the electronic device. In an operation 2106, gyroscopic (and/or accelerometer) data is used to determine the current orientation of the electronic device and associated medical device. At this point, the current orientation of the device may be compared with the designated orientation, for example, using an augmented reality-based user interface on the electronic device's display.

In another embodiment, 3D depth data may be captured using a stand-alone sensor system that incorporates some or all of the same sensors and components as, for example, sensory assembly 602 of electronic device 206 (see FIG. 6). Such a stand-alone sensor system may be packaged as a camera system that may be attached directly to an electronic device and/or an attachment apparatus and used as part of the alignment system.

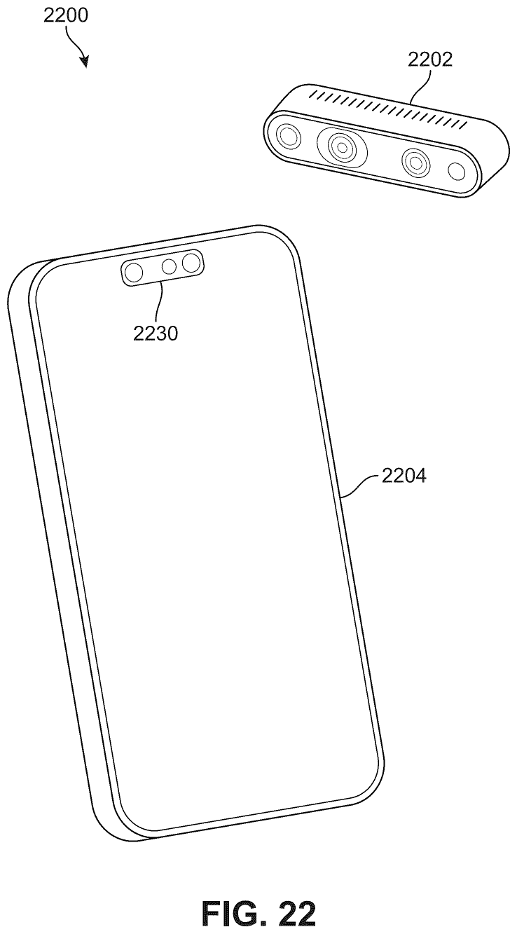

FIG. 22 is a schematic view of some components of an alignment system 2200, which includes an auxiliary sensor system 2202 and an electronic device 2204. Auxiliary sensor system 2202 (“sensor system 2202”) may include various sensors and components for capturing 3D depth data. In some cases, sensor system 2202 uses structured light to capture high resolution depth data. In some cases, sensor system 2202 includes one or more of a flood illuminator, an infrared camera, a dot projector, an RGB camera, as well as possibly a proximity sensor and an ambient light sensor.

Electronic device 2204 may include several common elements with earlier embodiments, such as a display, an IMU, a rear-facing RGB camera, and a LIDAR sensor. The device may also include software modules for processing data, such as registration modules, orientation modules, surgical planning modules, and augmented reality modules.

In some cases, electronic device 2204 may include a front-facing sensor assembly for gathering 3D depth data (such as sensor assembly 2230 in FIG. 22). However, because this sensor assembly is front-facing, it may not be oriented towards the surgical scene when the display is facing the surgeon. In such cases, additional data gathered from sensor system 2202 may be needed to supplement or replace the 3D depth data that might otherwise be gathered by the front-facing sensor assembly.

Sensor system 2202 may communicate with electronic device 2204 to provide the necessary 3D depth data for registration and other functions. This communication may occur wirelessly or through a wired connection, allowing the electronic device to receive and process the data from sensor system 2202 in real-time. In an exemplary embodiment, communication between sensor system 2202 and electronic device 2204 occurs wirelessly, for example, using a personal area network such as a Bluetooth network.

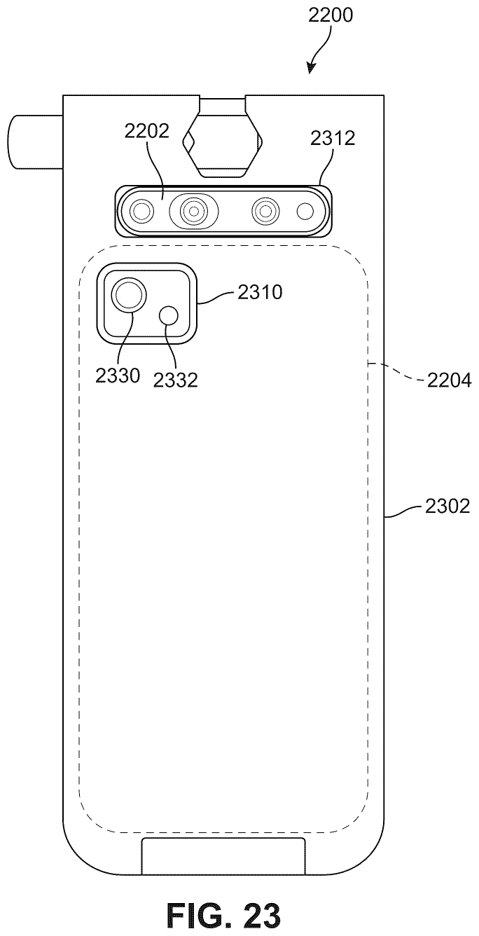

FIG. 23 shows another view of alignment system 2200, in which electronic device 2204 is enclosed within an attachment apparatus 2302. As shown in FIG. 23, a rear side of attachment apparatus 2302 includes a first rear opening 2310 associated with an RGB camera 2330 and a LIDAR sensor 2332. Additionally, the rear side of attachment apparatus 2302 includes a second rear opening 2312 through which sensor system 2202 is visible. That is, apparatus 2302 may be configured to hold sensor system 2202 such that sensor system 2202 faces out from the rear side of apparatus 2302. This allows sensor system 2202 to gather 3D depth data from the surgical scene as a surgeon is holding the alignment system with the display of the electronic device facing towards the surgeon.

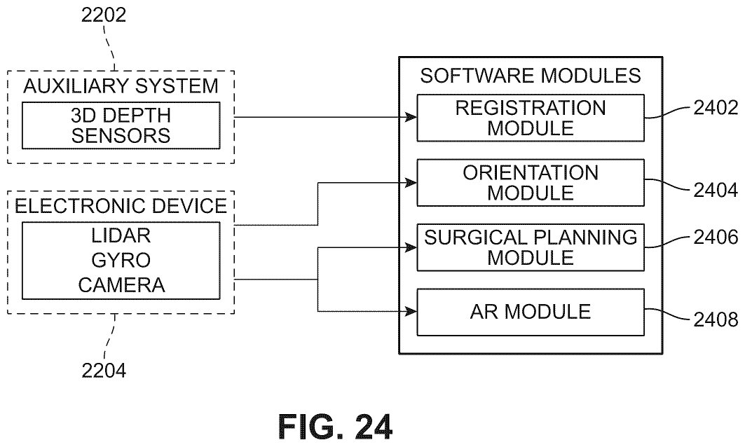

FIG. 24 shows a schematic view of the flow of data from different components of the alignment system to various software modules running on the electronic device. Referring to FIG. 24, the auxiliary sensor system 2202 includes 3D depth sensors (and associated components), which generate the necessary 3D depth data used by registration module 2402. The remaining data used by the software modules is all generated by components of electronic device 2204. These include LIDAR data generated by LIDAR sensors, IMU sensors (such as a gyroscope) and cameras. The data generated by the components of electronic device 2204 may be used by orientation module 2404, surgical planning module 2406, and AR module 2408.

Some embodiments may include provisions that facilitate providing visual guidance to a surgeon even when the display of the electronic device is oriented away from the surgeon. In some embodiments, the exemplary alignment system may use a secondary display device to provide visual guidance. As used herein, a “secondary display device” comprises any device with a display where the secondary display device is separate from the electronic alignment device. Moreover, the display of the secondary display device (or simply “display device”) is distinct from the display of the electronic alignment device.

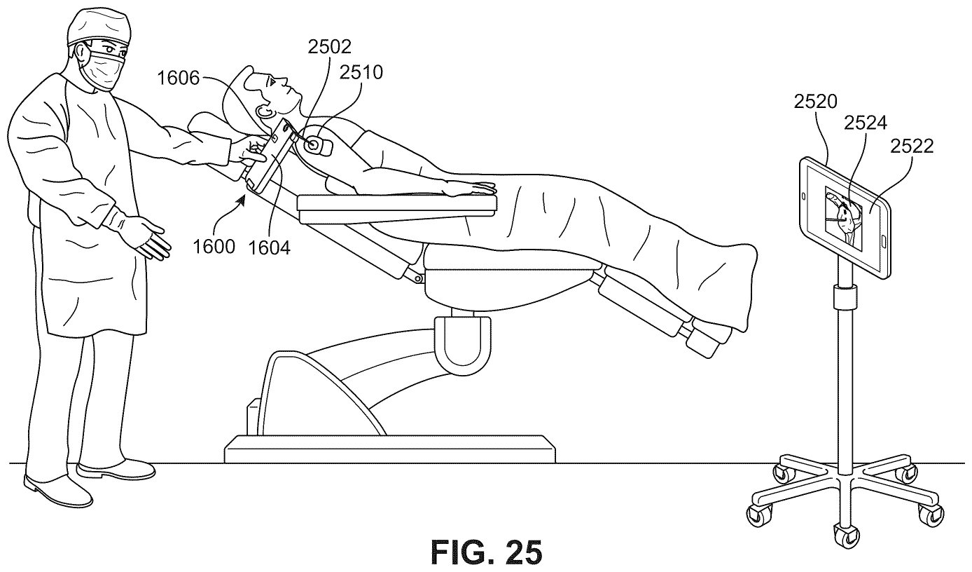

FIG. 25 depicts a schematic view of a scenario in which a surgeon is using alignment system 1600 to guide placement and insertion of a medical device 2502 into a patient's shoulder. Alignment system 1600 may include apparatus 1604 and electronic device 1606 (visible in FIG. 25 only through the rear camera opening). As seen in FIG. 25, in this scenario, the surgeon holds assembly 1604 such that a set of sensors of the electronic device for detecting 3D depth data are oriented towards the target area surgical 2510. In this case, the display of the electronic device is also oriented towards target surgical area 2510 and thus not visible to the surgeon. To facilitate guiding medical device 2502 while the display of the electronic device is oriented away from the surgeon, the embodiment includes a secondary display device 2520, which includes display 2522. In this case, an image 2524 of target surgical area 2510 is displayed along with augmented reality elements (as in the embodiment of FIG. 15 for example) to provide visual guidance for placement and/or alignment of medical device 2502.

It may be appreciated that display device 2520 may be any suitable kind of display device. In the embodiment of FIG. 25, display device 2520 is depicted as a table computing device (for example, an iPad) held on a roll-away stand. However, in other embodiments other suitable display devices may include monitors or screens mounted on carts or arms within the operating room (OR), integrated OR wall displays, laptops, computer monitors, smartphones, or other handheld devices capable of rendering graphical user interfaces. In still other embodiments, other suitable display devices may comprise headsets, such as virtual reality (VR) or augmented reality (AR) headsets. For example, a surgeon could wear an AR headset to see a live feed of the surgical environment with various guidance information displayed over the live feed using AR elements.

The guidance information displayed on the display device may include graphical indicators, overlays, or augmented reality (AR) content highlighting anatomical landmarks, recommended instrument trajectories, or proximity warnings. The selection and configuration of display devices may vary based on clinical workflow, available infrastructure, and user preference, with each capable of rendering static or dynamic content derived from preoperative planning data, intraoperative imaging, or real-time tracking inputs.

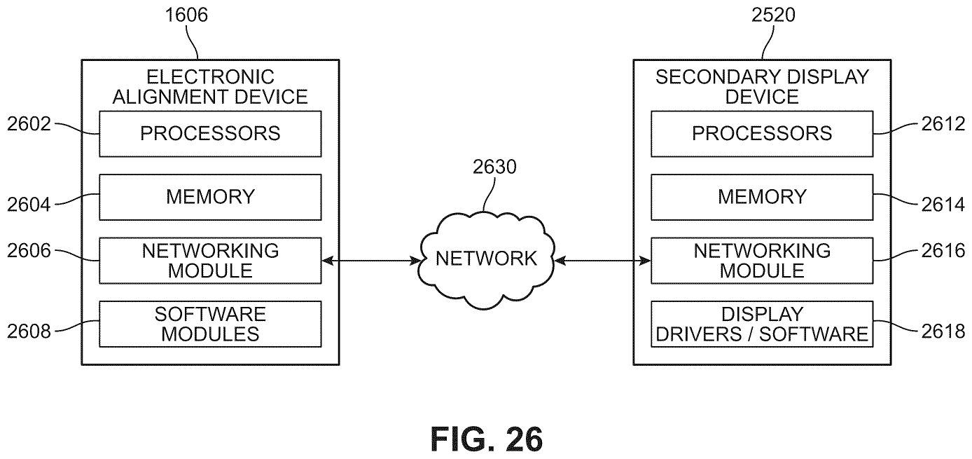

It may be appreciated that visual information provided on a secondary display device may be generated by the electronic alignment device. As an example, FIG. 26 depicts an architecture for communicating information between electronic alignment device 1606 and display device 2520. As part of this architecture, each component may be configured with corresponding processors and memory (for example, processors 2602 and memory 2604 for device 1606 and processors 2612 and memory 2614 for device 2520). Additionally, both devices may be configured with networking modules for communicating data, including networking module 2606 for device 1606 and networking module 2616 for device 2520. Using these network modules, device 1606 and device 2520 may communicate over a network 2630.

In addition, device 1606 may include suitable software modules 2608 that facilitate surgical planning, entry point selection, anatomical mapping and registration, alignment and visual guidance, as discussed above and shown, for example, in FIG. 11. Secondary display device 2520 may include display drivers and/or software 2618 for displaying visual data (including static images and/or video) provided by electronic device 1606.

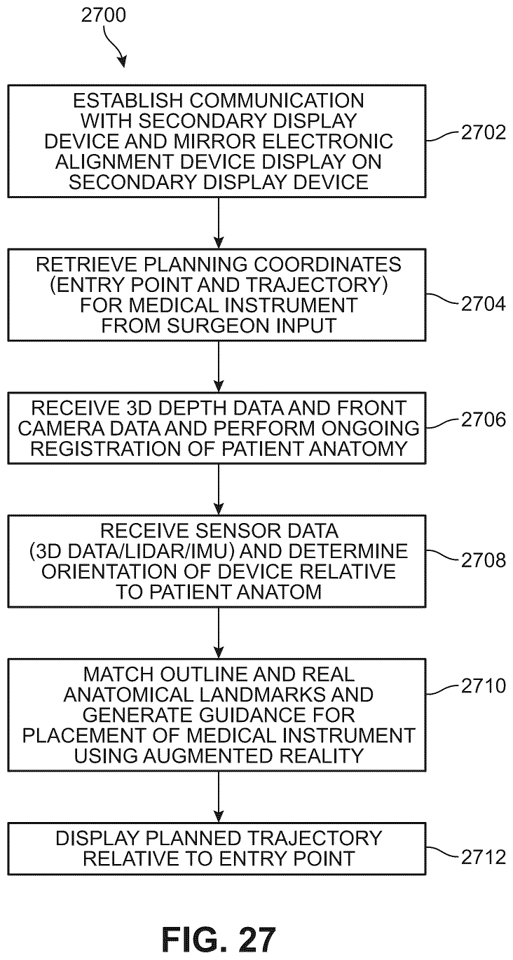

FIG. 27 illustrates a process for using the exemplary alignment system to perform a surgical procedure, such as total shoulder arthroplasty, according to an embodiment. Starting in step 2702, an electronic alignment device (such as device 1606) may establish communication with a secondary display device (such as device 2520) and send display data that allows the secondary display device to mirror the electronic alignment device's own display. As already mentioned, this allows a surgeon to view visual guidance information on the secondary display when the electronic alignment device's own display is oriented away from the surgeon.

In step 2704, the electronic device may receive planning coordinates that have been previously determined with a surgeon's input. The planning coordinates may include an entry point location and a trajectory for a medical instrument, such as a surgical guide pin or wire. The planning coordinates may be determined using any suitable method including the steps already described, for example, in process 100 of FIG. 1 or process 1200 of FIG. 12. In step 2706, once a surgeon has oriented the front facing 3D depth sensors of the device towards the target surgical area, the electronic device may receive 3D depth data and front camera data. The electronic device may process this data to perform ongoing registration of the anatomy in the target area (for example, the glenoid), as discussed above.

In step 2708, the electronic device may receive sensor data from any suitable combination of sensors in order to determine an orientation of the electronic device relative to the target anatomy. As already discussed, the system may receive data from sensors detecting 3D depth data, from optical cameras, from components of an Internal Measurement Unit (including a gyroscope, accelerometer, and/or magnetometer), from LIDAR sensors, or from any other suitable combination of sensors. The system may process this data to determine an orientation of the electronic device (and thus the corresponding orientation of the medical instrument that is coupled to the electronic device via the apparatus).

In step 2710, the electronic device, possibly in cooperation with the surgeon's manual adjustment of the device's position, may match the outline of the target anatomy with real anatomical landmarks (such as features of the glenoid). Based on this matching, the electronic device may generate guidance for helping the surgeon to place the medical instrument at the desired entry point using augmented reality. For example, the electronic device could generate a virtual element depicting the target entry point and overlay the virtual element onto an image of the target anatomy. As mentioned, because the secondary display device is mirroring the electronic device's own display, the virtual elements and underlying live images may be displayed on the secondary display device as well for convenient viewing by the surgeon.

Once the instrument is placed in the correct entry point location, the electronic device may display the planned trajectory relative to the entry point in step 2712. For example, a virtual instrument in the planned orientation may be overlaid with a feed of the target anatomy and the instrument so that the surgeon can place and align the instrument. Moreover, this information may be displayed on the secondary display device at the same time.

Various alternative embodiments are also contemplated.

In some embodiments, the system may not use augmented reality for guidance but may instead use other information or indicia displayed on the electronic device to provide feedback and guidance for the surgeon as they adjust the position and orientation of the medical device. For example, the display may show numerical values representing the current orientation of the medical device relative to the designated orientation. These values may be updated in real-time as the surgeon moves the device, allowing for precise adjustments.

In some cases, the system may use graphical indicators such as arrows or directional cues to guide the surgeon towards the desired orientation. The size or color of these indicators may change as the medical device approaches the target position and orientation. Additionally, the system may employ a crosshair or bullseye-type display, where the surgeon aims to align a central point with a target zone representing the desired entry point and trajectory.

The electronic device may also provide audio feedback to guide the surgeon. For instance, the frequency or volume of a tone may change as the medical device gets closer to or further from the designated orientation. In some implementations, the system may use haptic feedback, causing the electronic device or attachment apparatus to vibrate with varying intensity or patterns to indicate proximity to the target orientation.

The software processes and methods of the embodiments described in this detailed description and shown in the figures can be implemented using any kind of computing system having one or more central processing units (CPUs) and/or graphics processing units (GPUs). The processes and methods of the embodiments could also be implemented using special purpose circuitry such as an application specific integrated circuit (ASIC). The processes and methods of the embodiments may also be implemented on computing systems including read only memory (ROM) and/or random access memory (RAM), which may be connected to one or more processing units. Examples of computing systems and devices include, but are not limited to: servers, cellular phones, smart phones, tablet computers, notebook computers, e-book readers, laptop or desktop computers, all-in-one computers, as well as various kinds of digital media players.

The processes and methods of the embodiments can be stored as instructions and/or data on non-transitory computer-readable media. The non-transitory computer readable medium may include any suitable computer readable medium, such as a memory, such as RAM, ROM, flash memory, or any other type of memory known in the art. In some embodiments, the non-transitory computer readable medium may include, for example, an electronic storage device, a magnetic storage device, an optical storage device, an electromagnetic storage device, a semiconductor storage device, or any suitable combination of such devices. More specific examples of the non-transitory computer readable medium may include a portable computer diskette, a floppy disk, a hard disk, magnetic disks or tapes, a read-only memory (ROM), a random access memory (RAM), a static random access memory (SRAM), a portable compact disc read-only memory (CD-ROM), an erasable programmable read-only memory (EPROM or Flash memory), electrically erasable programmable read-only memories (EEPROM), a digital versatile disk (DVD and DVD-ROM), a memory stick, other kinds of solid state drives, and any suitable combination of these exemplary media. A non-transitory computer readable medium, as used herein, is not to be construed as being transitory signals, such as radio waves or other freely propagating electromagnetic waves, electromagnetic waves propagating through a waveguide or other transmission media (e.g., light pulses passing through a fiber-optic cable), or electrical signals transmitted through a wire.

Instructions stored on the non-transitory computer readable medium for carrying out operations of the present invention may be instruction-set-architecture (ISA) instructions, assembler instructions, machine instructions, machine dependent instructions, microcode, firmware instructions, configuration data for integrated circuitry, state-setting data, or source code or object code written in any of one or more programming languages, including an object oriented programming language such as Smalltalk, C++, python, java, or suitable language, and procedural programming languages, such as the “C”programming language or similar programming languages.

Aspects of the present disclosure are described in association with figures illustrating flowcharts and/or block diagrams of methods, apparatus (systems), and computing products. It will be understood that each block of the flowcharts and/or block diagrams can be implemented by computer readable instructions. The flowcharts and block diagrams in the figures illustrate the architecture, functionality, and operation of possible implementations of various disclosed embodiments. Accordingly, each block in the flowchart or block diagrams may represent a module, segment, or portion of instructions. In some implementations, the functions set forth in the figures and claims may occur in an alternative order than listed and/or illustrated.

The embodiments may utilize any kind of network for communication between separate computing systems. A network can comprise any combination of local area networks (LANs) and/or wide area networks (WANs), using both wired and wireless communication systems. A network may use various known communications technologies and/or protocols. Communication technologies can include, but are not limited to: Ethernet, 802.11, worldwide interoperability for microwave access (WiMAX), mobile broadband (such as CDMA, and LTE), digital subscriber line (DSL), cable internet access, satellite broadband, wireless ISP, fiber optic internet, as well as other wired and wireless technologies. Networking protocols used on a network may include transmission control protocol/Internet protocol (TCP/IP), multiprotocol label switching (MPLS), User Datagram Protocol (UDP), hypertext transport protocol (HTTP), hypertext transport protocol secure (HTTPS) and file transfer protocol (FTP) as well as other protocols.

Data exchanged over a network may be represented using technologies and/or formats including hypertext markup language (HTML), extensible markup language (XML), Atom, JavaScript Object Notation (JSON), YAML, as well as other data exchange formats. In addition, information transferred over a network can be encrypted using conventional encryption technologies such as secure sockets layer (SSL), transport layer security (TLS), and Internet Protocol security (Ipsec).

While various embodiments of the invention have been described, the description is intended to be exemplary, rather than limiting, and it will be apparent to those of ordinary skill in the art that many more embodiments and implementations are possible that are within the scope of the invention. Accordingly, the invention is not to be restricted except in light of the attached claims and their equivalents. Also, various modifications and changes may be made within the scope of the attached claims.

We claim:

1. A method, comprising:
receiving, at an electronic device, a designated trajectory for a medical device relative to an anatomical structure;
receiving, at the electronic device, 3D depth data from one or more sensors of the electronic device;
registering, using the 3D depth data, the electronic device to the anatomical structure;
receiving, at the electronic device, sensor information from a sensor that is different than the one or more sensors;
determining, using the sensor information, an orientation of the electronic device relative to the anatomical structure;
establishing communication between the electronic device and a display device that is separate from the electronic device;
sending, by the electronic device, image information to the display device, wherein the image information comprises information to guide a surgeon in aligning the medical device with the designated trajectory.

2. The method according to claim 1, wherein the electronic device includes a display and wherein the one or more sensors are on the same side of the electronic device as the display.

3. The method according to claim 1, wherein the sensor information comprises gyroscopic information.

4. The method according to claim 1, wherein the sensor information comprises magnetic information.

5. The method according to claim 1, wherein the sensor information comprises LIDAR information.

6. The method according to claim 1, wherein the image information includes augmented reality information.

7. An attachment apparatus for use with an electronic device for alignment of a medical device, the attachment apparatus comprising:
a first wall and a second wall opposite the first wall;
a cavity between the first wall and the second wall for receiving the electronic device;
a coupling apparatus for coupling the attachment apparatus to the medical device;
an opening on the second wall; and
a reflector assembly for redirecting light entering through the opening.

8. The attachment apparatus according to claim 7, wherein the first wall of the attachment apparatus includes a second opening, and wherein the second opening is configured so that a display of the electronic device is visible through the second opening when the electronic device is disposed within the cavity.

9. The attachment apparatus according to claim 7, wherein the reflector assembly redirects light entering through the opening to a location within the cavity associated with a sensor of the electronic device.

10. The attachment apparatus according to claim 9, wherein the sensor is used to gather 3D depth data.

11. The attachment apparatus according to claim 7, wherein the second wall includes a second opening, and wherein the second opening is configured to be aligned with a sensor of the electronic device when the electronic device is disposed within the attachment apparatus.

12. The attachment apparatus according to claim 11, wherein the sensor is an RGB camera.

13. The attachment apparatus according to claim 11, wherein the sensor is a LIDAR sensor.

14. The attachment apparatus according to claim 7, wherein the reflector assembly comprises at least two mirrors configured to redirect light entering through the opening.

15. A method, comprising:
receiving, at an electronic device, a designated trajectory for a medical device relative to an anatomical structure;
receiving, at the electronic device, 3D depth data;
registering, using the 3D depth data, the electronic device to the anatomical structure;
receiving, at the electronic device, gyroscopic information;
determining, using the gyroscopic information, an orientation of the electronic device relative to the anatomical structure; and
displaying, using the electronic device, information to guide a surgeon in aligning the medical device with the designated trajectory.

16. The method according to claim 15, wherein receiving the 3D depth data comprises receiving the 3D depth data from a first sensor array on a first side of the electronic device.

17. The method according to claim 16, wherein displaying information to guide the surgeon comprises displaying information on a display of the electronic device, wherein the display is disposed on a second side of the device disposed opposite of the first side of the electronic device.

18. The method according to claim 15, wherein the method further comprises sending information to guide the surgeon to a secondary device that is different from the electronic device.

19. The method according to claim 15, wherein the method further comprises receiving acceleration information at the electronic device and using both the gyroscopic information and the acceleration information to determine the orientation of the electronic device.

20. The method according to claim 15, wherein the method further comprises assisting the surgeon in creating the designated trajectory using the electronic device.
